(19) United States
(12) Patent Application Publication
(10) Pub. No.: US 2024/0044917 A1
(43) Pub. Date: Feb. 8, 2024

(54) METHOD FOR THE ASSESSMENT OF PERIMENOPAUSE OR MENOPAUSE STATUS VIA ANALYSIS OF THE IgG GLYCOME

(71) Applicants: GENOS d.o.o., Osijek (HR); GlycanAge LTD, Ruislip (GB)

(72) Inventors: Gordan LAUC, Zagreb (HR); Cristina MENNI, London (GB); Domagoj KIFER, Zagreb (HR); Nikolina LAUC, London (GB)

(73) Assignees: GENOS d.o.o., Osijek (HR); GlycanAge LTD, Ruislip (GB)

(21) Appl. No.: 18/373,311

(22) Filed: Sep. 27, 2023

Related U.S. Application Data
(63) Continuation of application No. PCT/EP2022/058071, filed on Mar. 28, 2022.

(30) Foreign Application Priority Data
Mar. 30, 2021 (HR) P20210509A
Mar. 30, 2021 (HR) P20210511A

(51) Int. Cl. G01N 33/68; G01N 33/58

(52) U.S. Cl. CPC G01N 33/6893; G01N 33/582; G01N 2800/362; G01N 2400/10

(57) ABSTRACT

The present disclosure reveals the diagnostic method for the determination of multiday average molar concentration of estradiol (E2) and from this parameter, the assessment of perimenopause and menopause status in examined female subjects, based on quantitative analysis of N-glycans bound on immunoglobulin G (IgG). The diagnostic process is applicable to the female subjects of 40-55 years of age. The revealed method enables the determination of the multiday average molar concentration of estradiol (E2), which is an important diagnostic parameter hardly accessible by any known single diagnostic method applied on only one or more blood analyses.

### CROSS-REFERENCE TO RELATED APPLICATIONS

The present application is a continuation of International Application No. PCI/1EP2022/058071, filed Mar. 28, 2022, which claims priority to HR P20210509A, filed Mar. 30, 2021, and HR P20210511A, filed Mar. 30, 2021, the entire disclosure of each are incorporated herein by reference.

### 1. TECHNICAL FIELD

The present invention discloses a method for the assessment of perimenopause and menopause status via the determination of the multiday average molar concentration of estradiol (E2) in female subjects of 40-55 years old, based on quantitative analysis of N-glycans bound to immunoglobulin G (IgG).

### 2. BACKGROUND

Glycans are complex carbohydrates predominantly based on N-acetyl-glucosamine (▪), fucose (∇), mannose (●), galactose (◯), and N-acetyl-neuraminic acid (♦), which are bound to proteins typically by N-glycoside bond, are involved in a plethora of physiological and pathological processes. Due to their influence on a large number of biological processes, they are recognized as important biochemical markers of general health and various physiological and pathological conditions of human organism; see literature reference 1:
1) G. Opdenakker, P. M. Rudd, C. P. Ponting, R. A. Dwek: Concepts and principles of glycobiology, FASEB J. 7 (1993) 1330-1337.

Immunoglobulin G (IgG) is the most represented antibody in the human plasma which exhibits an important role in defending organism from various pathogens. IgG is a glycoprotein for whose stability and function, the glycans bounded on its heavy chains are especially important. IgG glycosylation is also dependent on various physiological (age, sex, pregnancy) and pathological conditions (tumors, infections, autoimmune diseases). The changes in the IgG glycosylation pattern during aging are known in the art, and by monitoring IgG N-glycans, it is possible to derive the conclusion about the biological age of the examined subject; see literature references 2-5:
2) R. Parekh, I. Roitt, D. Isenberg, R. Dwek, T. Rademacher: Age-related galactosylation of the N-linked oligosaccharides of human serum IgG, J. Exp. Med. 167 (1988) 1731-1736.
3) M. Pu(id, A. Knezevid, J. Vidic, B. Adamczyk, M. Novokmet, O. Polasek, O. Gornik, S. Supraha-Goreta, M. R. Wormald, I. Redzid, H. Campbell, A. Wright, N. D. Hastie, J. F. Wilson, I. Rudan, M. Wuhrer, P. M. Rudd, D. Josid, G. Lauc: High Throughput Isolation and Glycosylation Analysis of IgG-Variability and Heritability of the IgG Glycome in Three Isolated Human Populations, Molecular & Cellular Proteomics 10.10; doi:10.1074/mcp.M111.010090.
4) EP3011335B1; G. Lauc, M. Pu(id-Bakovic, F. Vuckovid: Method for the analysis of N-glycans attached to immunoglobulin G from human blood plasma and its use; applicant: Genos d.o.o. (HR); priority date: 20.06.2013.
5) J. Kristic, F. Vuckovic, C. Menni, L. Klarid, T. Keser, I. Beceheli, M. Pucid-Bakovic, M. Novokmet, M. Mangino, K. Thaqi, P. Rudan, N. Novokmet, J. Sarac, S. Missoni, I. Kolcid, O. Polasek, I. Rudan, H. Campbell, C. Hayward, Y. Aulchenko, A. Valdes, J. F. Wilson, O. Gornik, D. Primorac, V. Zoldos, T. Spector, G. Lauc: Glycans are a novel biomarker of chronological and biological ages, J. Gerontol. A Biol. Sci. Med. Sci. 69 (2014) 779-789. doi: 10.1093/gerona/glt190.

Menopause is defined as a phase of the female life that starts 12 months after the last menstruation. It is characterized by complete or almost complete ovary exhaustion, which results in very low levels of female sex hormone estradiol (E2) in the serum, and significantly increased concentration of follicle-stimulating hormone (FSH).

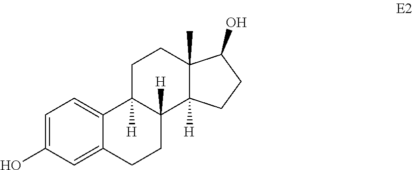

The transitional period from normal female fertile phase to the menopause onset is known as perimenopause. Common symptoms usually occur at around 47 years of age or 4-6 years before the menopause onset. The most often perimenopause symptoms are hot flushes, abnormal menstrual bleeding, insomnia, mood changes (anxiety, depression), mastodynia, headache, and vaginal dryness. Perimenopause is characterized by a decreased concentration of inhibin B, variable or increased FSH concentration, decreased AMH concentration and mildly decreased antral follicle number. These changes are accompanied with menstrual interval variation, decreased fertility and occurrence of said perimenopausal symptoms; see literature reference 6 and 7:
6) T. Hillard: NICE guideline—Menopause: diagnosis and management, Post Reprod. Health. 22 (2016) 56-58;
7) J. L. Bacon: The Menopause Transition, Obstet. Gynecol. Clin. N. Am. 44 (2017) 285-296.

Despite the fact that IgG N-glycans are changing with age, their connection with the perimenopause or menopause status has not been studied in detail. Especially it is not known whether their analysis could provide any conclusions about perimenopause or menopause onset.

Estradiol (E2) is a female sex hormone from the class of estrogens, which is useful as diagnostic marker for clinical estimation of diseases such as hypogonadism, hirsutism, polycystic ovary syndrome (PCOS), amenorrhea, ovarian cancer, for the monitoring of the therapy with aromatase inhibitors in female subjects, as well as for the control of fertility increasing therapies; see literature reference 8:
8) H. Ketha, A. Girtman, R. J. Singh: Estradiol assays—The path ahead, Steroids 99 (2015) 39-44.

The present invention solves the technical problem of a reliable method for determining average multiday estradiol (E2) concentration and, secondary, whether the examined female subject has entered into the perimenopause or menopause phase. It is known in the art that perimenopause or early menopause is hardly diagnosed due to significant day-to-day variations of sex hormones such as estradiol (E2), while the analysis of other known biochemical markers like follicle-stimulating hormone (FSH), anti-Müllerian hormone (AMH), or inhibin A or B are not conclusive enough.

Also, the determination of the multiday average molar concentration of estradiol (E2), which is an important diagnostic parameter, is not possible by any known single diagnostic method applied to only one or more blood or urine analyses.

The present disclosure solves this technical problem by quantitative analysis of N-glycans bound to immunoglobulin G (IgG) on the basis of one or more blood analyses.

### SUMMARY

The present invention discloses a method for the assessment of perimenopause and menopause status in female subjects by an analysis of N-glycans (I), bound to immunoglobulin G (IgG),

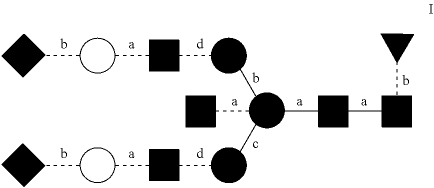

where the following symbols are used to denote chemical moieties:
▪ N-acetylglucosamine
▾ fucose
● mannose
◯ galactose
♦ N-acetylneuraminic acid

where letters a-d determine the type of glycoside bond of N-glycans (I):
a=β<1-4>
b=α<1-6>
c=α<1-3>
d=β<1-2>

via the determination of the multiday average concentration of estradiol (E2) from this IgG N-glycans analysis:

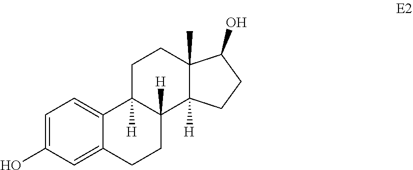

The said method comprises the following steps:
a) isolation of plasma from one or more blood samples that have been collected from the female subject under examination,
b) the release of said glycans from IgG,
c) quantitative analysis of thus released glycans in the free form or derivatized by fluorescent derivatization,
d) where the results from step c) are inserted in one or more numerical models suitable for the quantitative analysis used, where the said models are the result of statistical data analysis performed in the study that determined the variation of quantitative IgG glycans content in the blood plasma of a female cohort that was not in the menstruation phase or any other known medical condition associated with sex hormones fluctuations, and where the selected model gives numerical data indicative of multiday average estradiol (E2) molar concentration in the blood of the examined female subject, where the final result of this procedure provides a conclusion whether the examined female subject entered into perimenopause or menopause.

The method according to the present invention is applicable to female subjects between 40-55 years of age.

The method in step b) is performed by chemical or enzymatic means, most preferably with enzyme peptide-N4-(N-acetyl-beta-glucosaminyl)asparagine amidase F (PNGase F), and the quantitative analysis in step c) is performed with ultra-performance liquid chromatography (UPLC), MALDI-TOF mass spectrometry, coupled liquid chromatography and mass spectrometry (LC-MS), or capillary electrophoresis (CE).

The method according to the present invention enables the determination:
(i) of an average multiday molar concentration of estradiol (E2) in the blood for a 3-month period, preferably a 2-month period, and most preferably a 1-month period;
(ii) whether the examined female subject has passed through perimenopause and entered into menopause; or
(iii) whether the examined female subject has entered into perimenopause.

### BRIEF DESCRIPTION OF DRAWINGS

In order to explain the technical features of embodiments of the present disclosure more clearly, the drawings used in the present disclosure are briefly introduced as follow. Obviously, the drawings in the following description are some exemplary embodiments of the present disclosure. Ordinary person skilled in the art may obtain other drawings and features based on these disclosed drawings without inventive efforts.

FIG. 1 represents a typical chromatogram of 2-aminobenzamide (2AB) derived IgG N-glycans obtained by the ultra-high performance liquid chromatography (HILIC-UPLC) by the method described in Example 1, with 24 separated chromatographic peaks which are further in the text designed as GPB1-GPB24 according to one embodiment of the present disclosure.

FIG. 2 reveals the distribution of female subjects included in the study described in Example 2. N (female subjects)=70 according to one embodiment of the present disclosure.

FIG. 3 reveals the variability of IgG glycan properties for each examined female subject. Black vertical lines represent the scope of variability defined with the lowest and the highest level of agalactosylated (G0), monogalactosylated (G1), digalactosylated (G2), sialylated (S) and fucosylated (F) glycans, as well as glycans with bisecting GlcNAc (B) in total IgG N-glycome for each female subject during 12 weeks of the study duration; see Example 2 according to one embodiment of the present disclosure. Dashed vertical lines represent the variability scope of the same glycan properties in the control sample (standard).

FIG. 4 shows the model of the menstrual cycle according to one embodiment of the present disclosure. The use of the model menstrual cycle for the determination of the dynamic of the main glycan structure GPB4 from IgG N-glycome. N (sample)=500.

FIG. 5 shows the dynamics of IgG N-glycosylation during the menstrual cycle according to one embodiment of the present disclosure. Black curve describes the levels of six (6) derived IgG N-glycan properties: agalactosylated (G0), monogalactosylated (G1), digalactosylated (G2), sialylated (S), bisecting GlcNAc (B) and core fucose (F) during a few subsequent menstrual cycles. Standardised glycan measurements enable comparable variability of different derived IgG glycan properties. Each point represents one sample. N (samples)=500.

FIG. 6 shows the dynamics of sex hormones and IgG N-glycosylation in the menstrual cycle according to one embodiment of the present disclosure. The black curve describes the levels of six (6) derived IgG N-glycan properties: agalactosylated (G0), monogalactosylated (G1), digalactosylated (G2), sialylated (S), bisecting GlcNAc (B) and core-fucosylated (F) glycans, during a few subsequent menstrual cycles. Standardized glycan measurements enable comparable variability of different derived IgG glycan properties. Each point represents one sample. N (samples)=500.

### DETAILED DESCRIPTION

The present invention discloses a method for the assessment of perimenopause and menopause status in female subjects by an analysis of N-glycans (I), bound to immunoglobulin G (IgG) of general formula

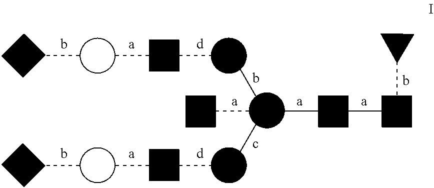

where the following symbols are used to denote chemical moieties:
▪ N-acetylglucosamine
▾ fucose
● mannose
◯ galactose
♦ N-acetylneuraminic acid

where letters a-d determine the type of glycoside bond of N-glycans (I):
a=β<1-4>
b=α<1-6>
c=α<1-3>
d=β<1-2>

via the determination of multiday average concentration of estradiol (E2) from this IgG N-glycans analysis:

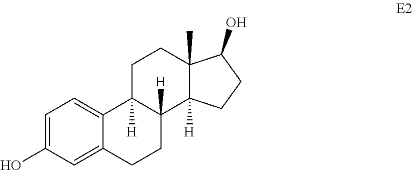

wherein the said method comprises the following steps:
a) isolation of plasma from one or more blood samples that have been collected from the female subject under examination,
b) the release of said glycans from IgG,
c) quantitative analysis of thus released glycans in the free form or derivatized by fluorescent derivatization,
d) where the results from step c) are inserted in one or more numerical models suitable for the quantitative analysis used, where the said models are the result of statistical data analyses performed in the study that determined the variation of quantitative IgG glycans content in the blood plasma of a female cohort that was not in the menstruation phase or any other known medical condition associated with sex hormones fluctuations, and where the selected model gives a numerical data indicative of the multiday average estradiol (E2) molar concentration in the blood of the examined female subject, where the final result of this procedure provides a conclusion whether the examined female subject entered into perimenopause or menopause.

The method from the present invention is applicable to female subjects between 40-55 years old.

The release of glycans I from IgG in step b) is performed by chemical or enzymatic means, most preferably with enzyme peptide-N4-(N-acetyl-beta-glucosaminyl)asparagine amidase F (PNGase F).

The method according to the present invention uses the quantitative analysis in step c) which is performed with ultra-performance liquid chromatography (UPLC), MALDI-TOF mass spectrometry, coupled liquid chromatography, and mass spectrometry (LC-MS), or capillary electrophoresis (CE).

The method according to the present invention uses the set of glycans I, which, upon release from IgG, are fluorescently derivatized in the step c) with a combination of:
(a) suitable aromatic amine such as 2-aminobenzamide (2AB), or other similar fluorescent dye, and
(b) suitable reducing agent for reductive amination like complex of picoline borane (BH₃·NC₅H₄-2-CH₃) or sodium cyanoborohydride (NaBH₃CN):

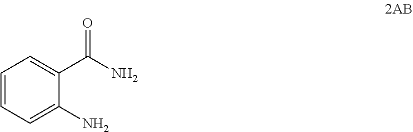

and the resulting mixture is analyzed by ultra-performance liquid chromatography (UPLC) for glycans GPB1-GPB24 as defined in Table 1:

Table 1. The set of immunoglobulin G (IgG) N-glycans that are released from IgG, and, after fluorescent derivatization with 2AB, analyzed on blood samples in order to determine the multiday average estradiol (E2) molar concentration in the examined female subject by the process from the present invention and, from this result, a conclusion whether the examined female subject entered into perimenopause or menopause.

| No | Glycans | Code | Structure |
|---|---|---|---|
| 1 | F(6)A1 | GPB1 | 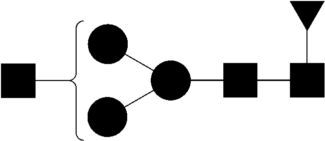 |
| 2 | A2 | GPB2 | 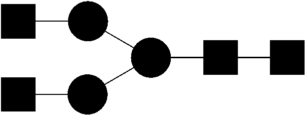 |
| 3 | A2B | GPB3 | 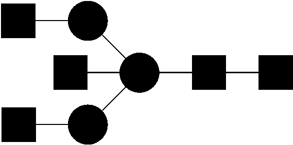 |
| 4 | F(6)A2 | GPB4 | 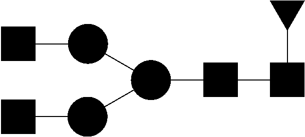 |
| 5 | M5 F(6)A2 | GPB5 | 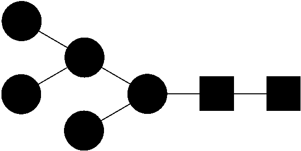 |
| | | | 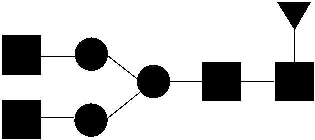 |
| 6 | F(6)A2B A2[6]G1 | GPB6 | 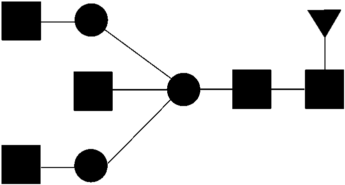 |
| | | | 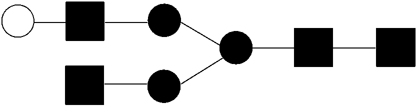 |
| 7 | A2[3]G1 F(6)A2B | GPB7 | 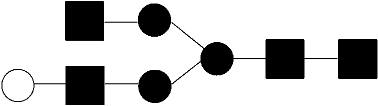 |
| | | | 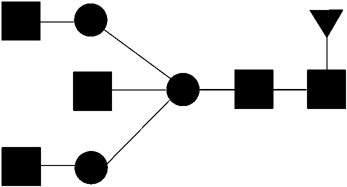 |
| 8 | A2BG1 F(6)A2[6]G1 | GPB8a | 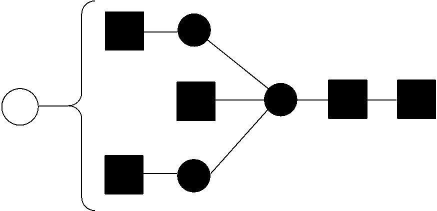 |
| | | | 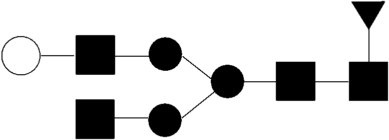 |
| 9 | F(6)A2[6]G1 | GPB8b | 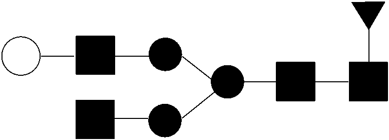 |
| 10 | F(6)A2[3]G1 | GPB9 | 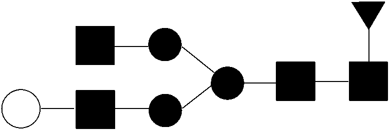 |
| 11 | F(6)A2[6]BG1 | GPB10 | 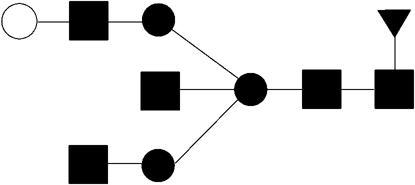 |
| 12 | F(6)A2[3]BG1 | GPB11 | 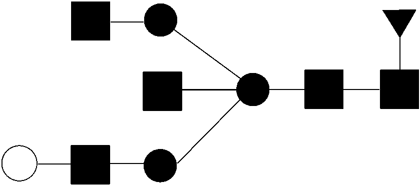 |
| 13 | A2G2 F(6)A2[3]BG1 | GPB12 | 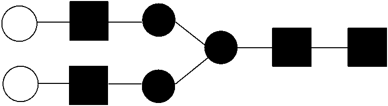 |
| | | | 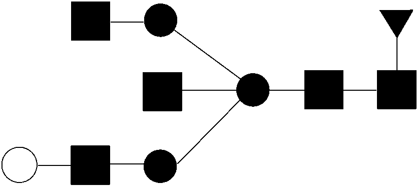 |
| 14 | A2BG2 F(6)A2G2 | GPB13 | 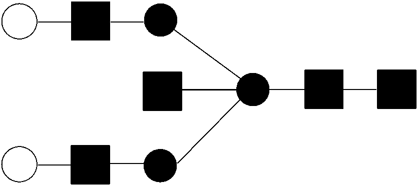 |
| | | | 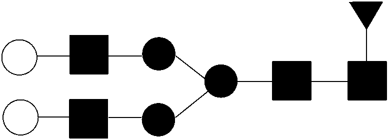 |
| 15 | F(6)A2G2 | GPB14 | 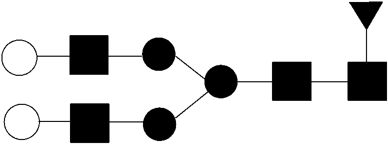 |
| 16 | F(6)A2BG2 F(6)A1G1S1 A2G1S1 F(6)A2G2 | GPB15 | 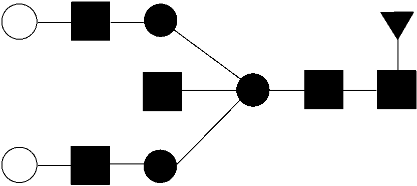 |
| | | | 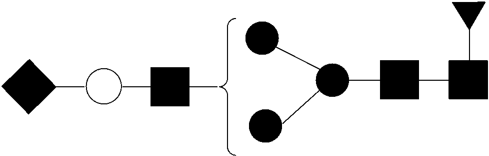 |
| | | | 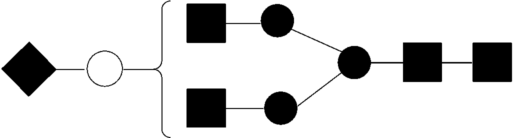 |
| | | | 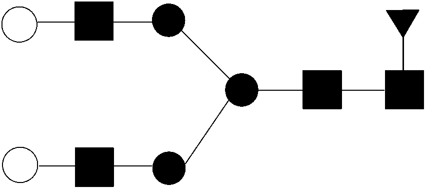 |
| 17 | F(6)A2[6]G1S1 M4A1G1S1 A2BG1S1 | GPB16a | 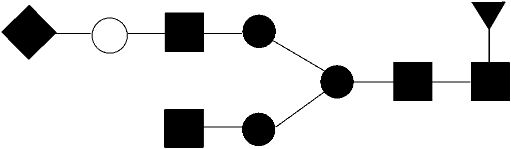 |
| | | | 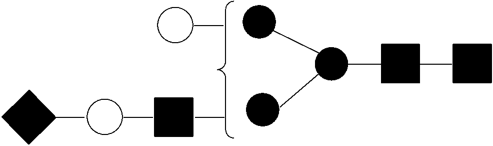 |
| | | | 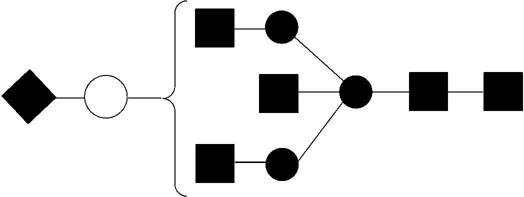 |
| 18 | F(6)A2[3]G1S1 F(6)A2[6]BG1S1 | GPB16b | 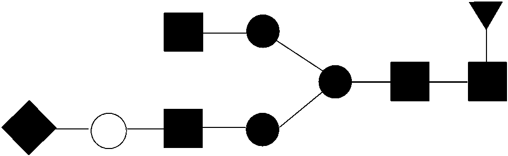 |
| | | | 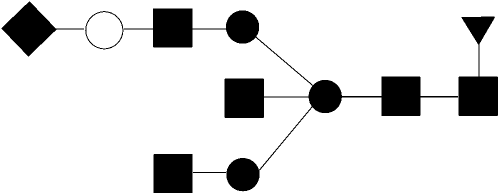 |
| 19 | A2G2S1 F(6)A2[3]BG1S1 | GPB17 | 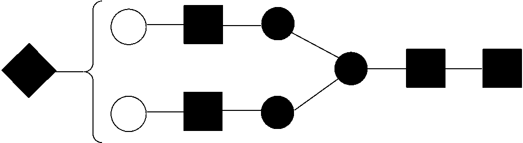 |
| | | | 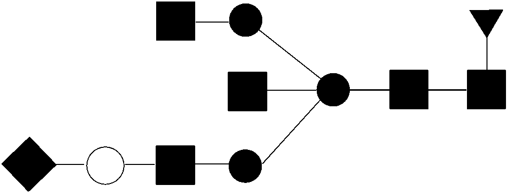 |
| 20 | A2BG2S2 F(6)A2G2S1 | GPB18a | 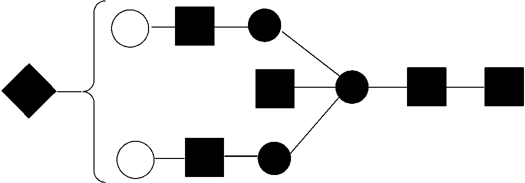 |
| | | | 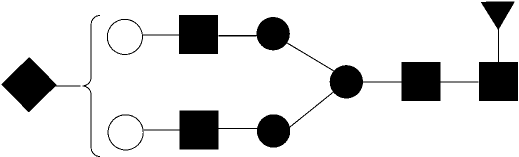 |
| 21 | F(6)A2G2S1 | GPB18b | 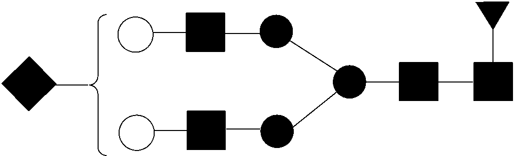 |
| 22 | F(6)A2BG2S1 | GPB19 | 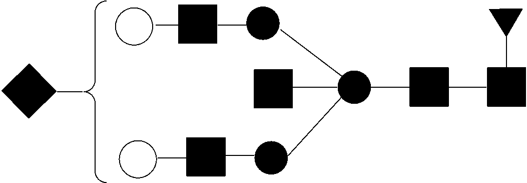 |
| 23 | — | GPB20 | * |
| 24 | A2G2S2 | GPB21 | 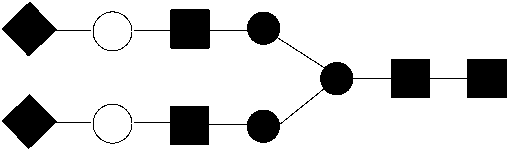 |
| 25 | A2BG2S2 | GPB22 | 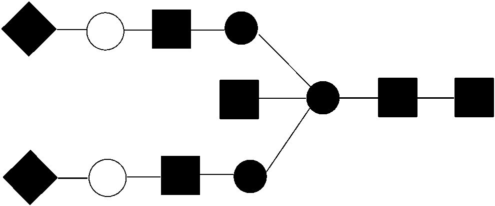 |
| 26 | F(6)A2G2S2 | GPB23 | 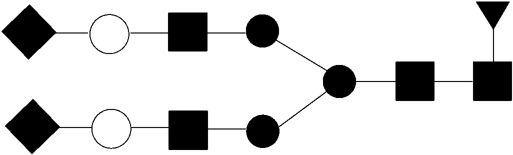 |
| 27 | F(6)A2BG2S2 | GPB24 | 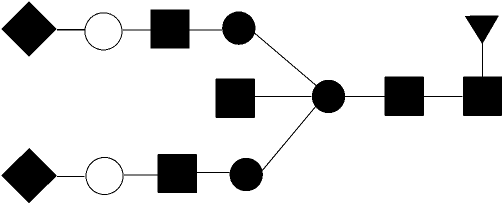 |

*The structure of glycan GPB20 was not determined, but was characterized by the retention time (t_{R}) in the respective analytical method.

From the determination of quantitative concentration of said glycans, the logarithm of multiday average molar concentration of estradiol (E2) is calculated from the following numerical model:

Log c(E2)=−15.529·GPR4−2.602·GPR8+5.589·GPR10+9.699·GPR12+53.911·GPR15+9.901·GPR16−1.990·GPR2·GPR10−0.065·GPR2·GPR12+3.601·GPR2·GPR15+0.007·GPR2·GPR16+0.465·(GPR4)²+2.889·GPR4·GPR8+5.106·GPR4·GPR10−0.817·GPR4·GPR12−8.606·GPB4·GPB15+1.490·GPB4·GPB18+1.689·(GPB8)²−9.048·GPB8·GPB10−0.999·GPB8·GPB12−2.253·GPB8·GPB15+3.143·(GPB10)²+0.712·GPB10·GPB12−3.505·GPB10·GPB15−4.753·GPB10·GPB16+1.128·GPB10·GPB18−4.584·GPB12·GPB15+1.138·GPB12·GPB16−1.355·GPB12·GPB18−0.598·GPB12·GPB22−0.904·GPB12·GPB23−4.638·(GPB15)²+0.287·GPB15·GPB16−3.049·GPB15·GPB18+2.492·(GPB16)²−3.041·GPB16·GPB18

wherein factors GPB2, GPB4, GPB8, GPB10, GPB12, GPB15, GPB16, GPB18, GPB22 and GPB23 represent natural logarithms of corresponding values belonging to relative areas under the peaks of the respective glycans GPB2, GPB4, GPB8, GPB10, GPB12, GPB15, GPB16, GPB18, GPB22, GPB23 obtained from the chromatogram given by the selected quantitative analytical technique, and from which the multiday average molar concentration of estradiol c(E2) is calculated and expressed in picomoles per liter (pmol/L).

Thus, the obtained result of the multiday average molar concentration of estradiol c(E2) is interpreted as follows:
(a) c(E2) from 7 to 80, then the female subject has passed through perimenopause phase and entered into menopause; or
(b) c(E2) from 80 to 800, then the female subject has not yet passed the perimenopause and thus not entered menopause.

Alternatively, according to the present invention, the set of glycans I, released from IgG, can be optionally fluorescently derivatized in the step c) with 5-dioxopyrrolidine-1-yl-[2N-(2-(N′,N′-diethylamino)ethyl)carbamoyl]-quinoline-6-yl-carbamate (RF):

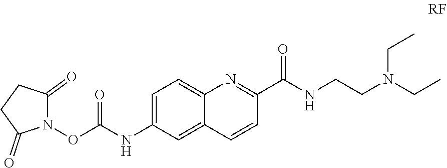 [SMILES: CCN(CC)CCNC(=O)c1ccc2cc(NC(=O)ON3C(=O)CCC3=O)ccc2n1]

or other similar fluorescent dye and the resulting mixture is analyzed, e.g., by ultra-performance liquid chromatography (UPLC).

The application of said derivatization reagents 2AB and RF was known from the prior art; see literature references 9 and 10:
9) T. Keser, T. Pavid, G. Lauc, O. Gornik: Comparison of 2-Aminobenzamide, Procainamide and RapiFluor-MS as Derivatizing Agents for High-Throughput HILIC-UPLC-FLR-MS N-glycan Analysis, Front. Chem. 6 (2018) 321; doi: 10.3389/fchem.2018.00324.
10) GlycoWorks RapiFluor-MS N-glycan Kit—96 Samples; Waters Corporation, 34 Maple Street, Milford, MA 01757 (SAD); www.waters.com; see the hyperlink:
https://www.waters.com/waters/library.htm?cid=511436&lid=13483484 5&lcid=134834844&locale=en_US.

The process according to the present invention uses the quantitative analysis in step c) which is performed with ultra-performance liquid chromatography (UPLC), MALDI-TOF mass spectrometry, coupled liquid chromatography and mass spectrometry (LC-MS), or capillary electrophoresis (CE) or other suitable analytical technique. The description of the quantitative analysis of IgG N-glycans with different analytical techniques is known in the prior art; see literature reference 4.

Analytics of N-Glycans from Blood Plasma

The isolation of blood plasma from female subject for the purpose of menopause diagnostics is performed by the methodology described in the prior art; see literature reference 4. Also, the isolation of IgG from the blood plasma was conducted by the method described in the prior art; see literature reference 11:
11) I. Trbojevid Akmacid, I. Ugrina, G. Lauc: Comparative Analysis and Validation of Different Steps in Glycomics Studies, Methods Enzymol. 586 (2017) 37-55. doi: 10.1016/bs.mie.2016.09.027.

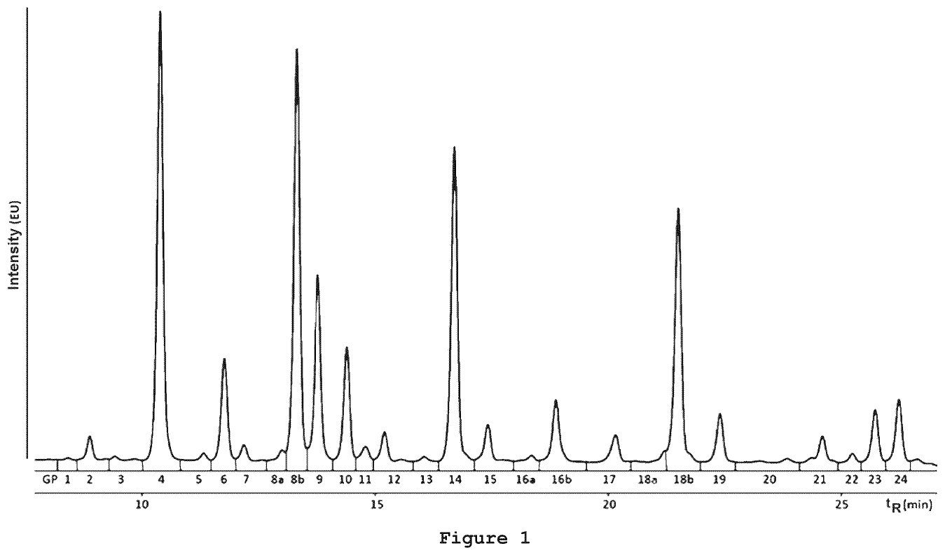

The typical chromatogram of 2-aminobenzamide (2AB) derived IgG N-glycans obtained by the UPLC method with 24 separated peaks, designed in Table 2 with abbreviations GPB1-GPB24 is shown in FIG. 1. The elution sequence of glycan peaks GPB1-GPB24 in said UPLC method and corresponding retention times (t_{R}) are presented in Table 2. This analytical method is disclosed in Example 1.

TABLE 2
Retention times (t_{R}) of 2-aminobenzamide (2AB) labelled IgG
glycans designed with codes GPB1-GPB24 obtained by the UPLC-
HILIC method described in Example 1 whose typical chromatogram
is shown in FIG. 1.

| No. | Glycan | t_{R} [min] | No. | Glycan | t_{R} [min] |
|---|---|---|---|---|---|
| 1 | GPB1 | 5.3 | 13 | GPB13 | 7.4 |
| 2 | GPB2 | 5.5 | 14 | GPB14 | 7.5 |
| 3 | GPB3 | 5.7 | 15 | GPB15 | 7.7 |
| 4 | GPB4 | 5.9 | 16 | GPB16 | 8.1 |
| 5 | GPB5 | 6.1 | 17 | GPB17 | 8.4 |
| 6 | GPB6 | 6.2 | 18 | GPB18 | 8.8 |
| 7 | GPB7 | 6.4 | 19 | GPB19 | 9.0 |
| 8 | GPB8 | 6.6 | 20 | GPB20 | 9.5 |
| 9 | GPB9 | 6.8 | 21 | GPB21 | 9.6 |
| 10 | GPB10 | 6.9 | 22 | GPB22 | 9.8 |
| 11 | GPB11 | 7.0 | 23 | GPB23 | 10.0 |
| 12 | GPB12 | 7.1 | 24 | GPB24 | 10.2 |

The study of variability of IgG N-glycans against the concentration of sex hormones and, subordinate, estradiol (E2) during different phases of the menstrual cycle in female subjects of 18-50 years and the development of the numerical model for the calculation of the multiday average estradiol (E2) concentration according to the present invention The study was performed on healthy adult female subjects of 18-50 years of age. Inclusion criteria were made on the data of previous menstrual cycles, health condition, and lifestyle of each subject obtained by elimination questionnaire. Inclusion criteria were age (18-50 years) and regular and normal menstrual cycles; see literature reference 12:
12) M. Mihm, S. Gangooly, S. Muttukrishna: The normal menstrual cycle in women, Anim. Reprod. Sci. 124 (2011) 229-236.

Additionally, in the study were included only those female subjects that did not have any diagnosed diseases that are connected with any known changes in IgG N-glycome pattern, such as inflammatory diseases, autoimmune diseases, various infections, and cancers; see literature reference 13:
13) I. Gudelj, G. Lauc, M. Pezer: Immunoglobulin G glycosylation in aging and diseases, Cell. Immunol. 333 (2018) 65-79.

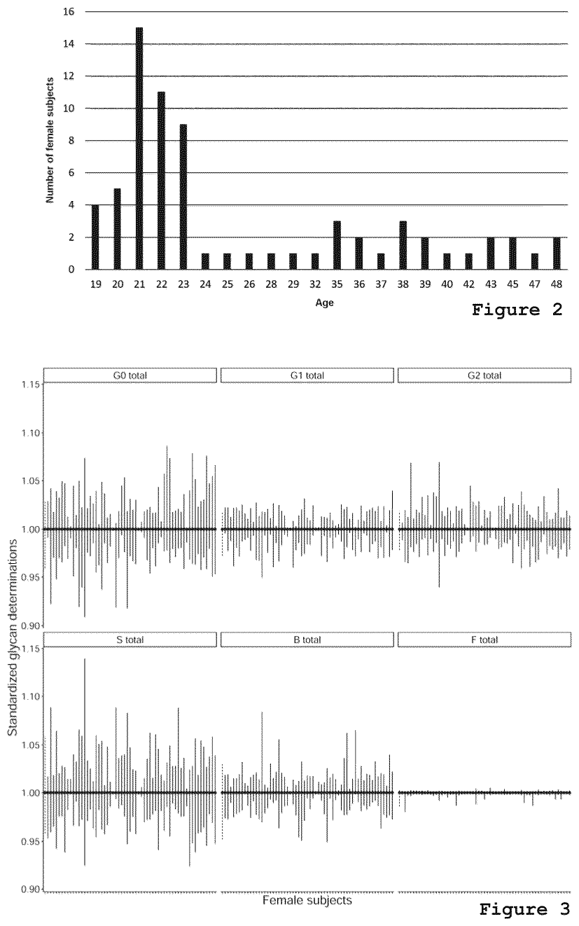

Exclusion criteria were: pregnancy, breastfeeding, menopause, use of oral contraceptives, use of other hormonal drugs, smoking and alcohol consumption. There were 70 healthy adult female subjects included in the study, all in the range from 19-48 years of age; see FIG. 2.

The Protocol of the Study

To analyze IgG N-glycosylation, the samples of blood plasma were collected. Sampling was performed in September to November 2016, during twelve (12) subsequent weeks, once a week (in the morning), with regular seven-days intervals and independently of the menstrual period of each particular woman. Detailed procedure for blood collection is described in Example 1.

Immunochemical Method for the Determination of Sex Hormones Concentration

The concentrations of sex hormones estradiol (E2), progesterone (P), and testosterone (T) were determined in plasma samples by microparticles-mediated chemiluminescence method (CMIA) on ARCHITECT® i1000SR (Abbott Diagnostics) automatized system by using commercially available reagent sets for the hormone determination, with exchangeable protocols of this manufacturer; see literature reference 14:
14) R. Stricker, R. Eberhart, M. C. Chevailler, F. A. Quinn, P. Bischof, R. Stricker: Establishment of detailed reference values for luteinizing hormone, follicle stimulating hormone, estradiol and progesterone during different phases of the menstrual cycle on the Abbott ARCHITECT analyzer, Clin. Chem. Lab. Med. 44 (2006) 883-887.

Detailed procedure for the quantitative analysis of sex hormones and, subordinately, estradiol (E2) is described in Example 2.

The analysis of IgG N-glycans in this study was also performed by the methodology known in the prior art; see literature reference 4. Also, the IgG isolation from human plasma was conducted by the known procedure; see literature references 3 and 11. The rest of experimental details are given in Example 2.

The Interpretation of the Study Results

Elimination of Variations of Glycan Data Due to Different Experiment Series

To decrease the variability due to experiment series (batch effect), all samples from the same female subject collected in twelve (12) time points, were analyzed on the same plate. On each plate there were randomly distributed plasma samples of maximally 3-5 subjects of approximately equal average age. The plates also contained the standard plasma sample in pentaplicate which served for the control of non-biological variability (technical variability of the method). Such methodology enabled variability between plates, and common correction of glycan data variability on series (batch correction) was thus avoided.

The Processing of the Glycan Data

Each chromatogram obtained during IgG N-glycans analysis was integrated and separated into 24 glycan peaks as shown in FIG. 1. Glycan data were firstly normalized on total glycan area (total chromatographic area). The area of each particular glycan peak was divided by total area of the corresponding chromatogram. This makes the measurements of different samples comparable. The amount of each N-glycan was expressed as a percentage of the total integrated area (% area); see literature reference 3. The set of about 20 manually integrated chromatograms was employed as a template for automatic integration of all IgG N-glycome chromatograms in this study; see literature reference 15:
15) A. Agakova, F. Vuĉković, L. Klarid, G. Lauc, F. Agakov: Automated Integration of a UPLC Glycomic Profile, Methods Mol Biol. 1503 (2017) 217-233.

Determination of Derived IgG Glycan Properties

Beside 24 directly determined glycan properties, 6 derived properties of IgG N-glycans were calculated. They separate the glycans against their particular structural characteristics for better analysis and understanding the glycan-involved biological processes; see Table 3. Said derived properties represent relative distribution of:
(i) galactosylated glycans: G0=glycans without galactose; G1=glycans with one (1) galactose molecule; G2=glycans with two (2) galactose molecules;
(ii) sialylated glycans: S=glycans with terminal sialic acid;
(iii) fucosylated glycans: F=glycans with core fucose; and
(iv) glycans with bisecting N-acetylglucosamine (GlcNAc): B=bisecting glycans; in total IgG glycome.

TABLE 3
Formulas for the calculation of derived IgG N-glycan properties.

| No. | Derived glycan properties | Chromatographic peaks |
|---|---|---|
| 1 | G0—agalactosylated | GPB1 + GPB2 + GPB3 + GPB4 + GPB6 |
| | glycans | |
| 2 | G1—monogalactosylated | GPB7 + GPB8 + GPB9 + GPB10 + GPB11 |
| | glycans | |
| 3 | G2—digalactosylated | GPB12 + GPB13 + GPB14 + GPB15 |
| | glycans | |
| 4 | S—sialylated glycans | GPB16 + GPB17 + GPB18 + GPB19 + GPB21 + GPB22 + |
| | | GPB23 + GPB24 |
| 5 | F—glycans with core | GPB1 + GPB4 + GPB6 + GPB8 + GPB9 + GPB10 + GPB11 + |
| | fucose | GPB14 + GPB15 + GPB16 + GPB18 + GPB19 + GPB23 + GPB24 |
| 6 | B—glycans with bisecting | GPB3 + GPB6 + GPB10 + GPB11 + GPB13 + GPB15 + GPB19 + |
| | GlcNAc | GPB22 + GPB24 |

The connection between sex hormones dynamic and IgG glycan properties in the menstrual cycle was examined. The time shift in sex hormones dynamic and IgG N-glycan properties is based on the comparison of their highest (peak) values during the menstrual cycle. The day within the menstrual cycle when the highest concentration of each sex hormone and the highest level of particular glycan property were observed, was calculated by the equation (1):

MC peak(X)=peak(X)×MC duration period (1)
wherein:
X₁, X₂∈X; X₁=sex hormone, X₂=glycan property; MC duration period=30 days (the average menstrual cycle period within the study); MC=menstrual cycle. Example:
MC peak (E2)=45%×30 days=0.45×30 days=13.5 13. day of MC
MC peak (S)=84%×30 days=0.84×30 days=25.2 25. day of MC

The time that has passed from the day when the highest concentration of each particular sex hormone was observed to the day when the highest level of each particular glycan property was detected, represents a time shift (MS shift) in the dynamic of said glycan properties during the menstrual cycle (MC), and is calculated according to the equation (2):

MC Shift(X₁,X₂)=MC peak(X₂)−MC peak(X₁) (2)

wherein:
X₁, X₂∈X; X₁=sex hormone, X₂=glycan property; MC duration period=30 days (the average menstrual cycle period within the study); MC=menstrual cycle. Example:
MC Shift (S, E₂)=MC peak (S)−MC peak (E₂)=25. day−13. day=12 days

Statistical Analysis of Obtained Results

The results were analyzed and visualized with the programming language R (version 3.0.1). Glycans and sex hormones dynamic within the menstrual cycle was approximated in a model menstrual cycle. The duration of the menstrual cycles in the study was standardized by dividing the duration of each menstrual cycle with 100%. This enables positioning and comparison of glycan data within a common model of menstrual cycle independently on the duration of each particular menstrual cycle. Glycan measurements were standardized by the dividing of each measurement result with its average value to enable comparison between different glycan properties.

The analysis of the connection of menstrual cycle with glycan properties was derived by the use of linear mixed model. Within this model, the fixed variable was age, while the random variable was the examined female subject. The assumed periodical pattern of longitudinal glycans measurements was modeled as a linear combination of sinusoidal and cosinusoidal function for menstrual cycle phases. In the said linear mixed model, the p values were corrected on multiple tests with the Benjamini-Hochberg method. p values lower than 0.05 were considered statistically significant.

Characteristics of Examined Female Subjects

During the inclusion of female subjects into the study, all general anthropometric, and health data connected with menstrual cycle were collected. The description of the included cohort is given in Table 4.

TABLE 4
Description of female subjects included in the study.

| No. | General data | | Anthropometric data | |
|---|---|---|---|---|
| 1 | Number of subjects | 70 | Height (cm) | 161.4 ± 4.1 |
| | (N) | | | |
| 2 | Nationality | Han Chinese | Weight (kg) | 56 ± 7.3 |
| 3 | Sex | female | Waist | 75.6 ± 7.5 |
| | | | circumference | |
| | | | (cm) N = 69 | |
| 4 | Age at sampling | 27.3 ± 8 | Hip | 95.1 ± 6.8 |
| | (years) | 19 | circumference | |
| | minimally: | 48 | (cm) N = 69 | |
| | maximally: | | | |
| 5 | Profession: | | WHtR (N = 69): | |
| | worker | 17 (24.3%) | malnourished | 2 (2.9%) |
| | student | 46 (65.7%) | healthy weight | 55 (79.8%) |
| | farmer | 5 (7.1%) | obese | 12 (17.3%) |
| | nurse | 2 (2.9%) | | |
| | retired | 0 | | |
| | others | 0 | | |
| 6 | Education degree: | | | |
| | Primary school | 1 (1.4%) | | |
| | lower secondary | 9 (12.9%) | | |
| | school | | | |
| | higher secondary | 6 (8.6%) | | |
| | school | | | |
| | faculty or higher | 54 (77.1%) | | |

| | Health data | | |
|---|---|---|---|
| 7 | Smoking history | NO | |
| 8 | Alcohol consumption history | NO | |
| 9 | Pregnancy | 0 | 47 (67.1%) |
| | (number per subject) | 1 | 11 (15.7%) |
| | | 2 | 5 (7.1%) |
| | | 3 | 6 (8.6%) |
| | | 4 | 1 (1.4%) |
| 10 | Childbirth | 0 | 47 (67.1%) |
| | (number per subject) | 1 | 20 (28.6%) |
| | | 2 | 3 (4.3%) |
| 11 | Miscarriage | 0 | 60 (85.7%) |
| | (number per subject) | 1 | 3 (4.3%) |
| | | 2 | 7 (10%) |

| | | |
|---|---|---|
| 12 | Appearance of the first | 13.3 ± 1 |
| | menstruation (year) | |
| 13 | Regular menstrual cycles | YES |
| 14 | Duration of menstrual cycle | 30 ± 5.8 |
| | (days) | |
| 15 | Duration of menstrual bleeding | 5.6 ± 1 |
| | (days) | |

Before inclusion into the study, the health status of female subjects was partially known. Particularly whether the subject is ill with acute or chronic disease, does not use hormone-replacing therapy, and is not pregnant or in menopause. In this manner, only healthy subjects were selected and included in the study, to avoid the potential influence of said health factors on IgG glycosylation; see literature reference 13. Continuous variables with normal distribution are shown as average values ±SD. Categorical variables are shown as percentages. WHtR (waist-to-height ratio) is used as a parameter of the body fat distribution in the abdominal part of the body; see literature reference 16:
16) Q. Ibrahim, M. Ahsan: Measurement of Visceral Fat, Abdominal Circumference and Waist-hip Ratio to Predict Health Risk in Males and Females, Pak. J. Biol. Sci. 22 (2019) 168-173.

Biological Variability of IgG N-Glycans

In order to study whether any changes in the IgG N-glycosylation occurred during the study, the eventual biological variability of each N-glycan was first determined. In this manner, in each plate, together with samples, one sample of known glycan profile (standard) was analyzed. Biological variability was then calculated as a ratio between the average variability values of a sample with a known glycan profile (standard) and the sample from the study for all 24 glycan peaks and multiplied by 100%. A ratio lower than 100% means that the biological variability of the analyzed glycan peak (glycan) is larger than the technical variability of the method. By comparison of glycan profiles of the standards with glycan profiles of the samples, it was discovered that the biological variability exceeds analytical variability in 14 of 24 IgG glycans monitored in the study; see Table 5 and FIG. 1. The glycan peaks that exhibit significant biological variability are marked underlined. The IgG N-glycan structures are shown in Table 1.

TABLE 5
Biological variability of particular IgG
N-glycans within the menstrual cycle.

| | | | Ratio |
|---|---|---|---|
| GPB | Standard | Sample | (%) |
| GPB1 | 0.01861 | 0.00885 | 210 |
| GPB2 | 0.00938 | 0.00756 | 124 |
| GPB3 | 0.00919 | 0.01186 | 77 |
| GPB4 | 0.00019 | 0.00038 | 50 |
| GPB5 | 0.06191 | 0.04149 | 149 |
| GPB6 | 0.0003 | 0.00086 | 35 |
| GPB7 | 0.00602 | 0.00543 | 111 |
| GPB8 | 0.00012 | 0.00017 | 71 |
| GPB9 | 0.00029 | 0.00055 | 53 |
| GPB10 | 0.00008 | 0.00019 | 42 |
| GPB11 | 0.00318 | 0.00508 | 63 |
| GPB12 | 0.00237 | 0.00168 | 141 |
| GP13 | 0.00567 | 0.0065 | 87 |
| GP14 | 0.00007 | 0.00022 | 32 |
| GP15 | 0.00065 | 0.00059 | 110 |
| GP16 | 0.00018 | 0.0005 | 36 |
| GP17 | 0.00319 | 0.00118 | 270 |
| GP18 | 0.00027 | 0.00058 | 47 |
| GP19 | 0.0024 | 0.00146 | 164 |
| GP20 | 0.01086 | 0.01977 | 55 |
| GP21 | 0.00599 | 0.00714 | 84 |
| GP22 | 0.02226 | 0.01479 | 151 |
| GP23 | 0.00186 | 0.00231 | 81 |
| GP24 | 0.00299 | 0.00191 | 157 |

GPB = glycan peaks GPB1-GPB24.

Variability of Derived IgG N-Glycan Properties

The variability of the derived IgG N-glycan properties was determined in the same manner as the biological variability of each particular glycan. The changing scope of glycosylation properties within the same subject was most profound for sialylated (the highest difference between the lowest and the highest value is about 21%) and agalactosylated (about 16%) glycans. Fucosylated glycans had the lowest intra-individual variability (lower than 3%), during the menstrual cycle; see FIG. 3.

For the analyzed cohort, average values were calculated values of the first and third quartile and minimal and maximal values of derived IgG glycan properties. The levels of each glycan property are shown in Table 6:

TABLE 6
The level of particular derived glycan properties (DGP) in
blood samples (n = 776) in analyzed cohort of female subjects (N = 70)
and control samples (n = 56) of the standard. Relative percentage of
galactosylation: G0 = agalactolysated, G1 = monogalactolysated, G2 =
digalactolysated; S = sialylated; B = bisecting GlcNAc; and F =
fucosylated; IgG in a total area of all glycan structures. Q1 = first
quartile (25. percentile), Q3 = third quartile (75. percentile).

| DGP | Cohort of female subjects | | | | | | Control samples | | | | | |
|---|---|---|---|---|---|---|---|---|---|---|---|---|
| (%) | min | Q1 | MV | MD | Q3 | max | min | Q1 | MV | MD | Q3 | max |
| G0 | 11.3 | 15.8 | 19.3 | 19.4 | 22.0 | 34.6 | 21.1 | 22.6 | 22.0 | 22.0 | 21.7 | 22.2 |
| G1 | 25 | 31.0 | 32.2 | 32.6 | 33.6 | 37.2 | 37.0 | 38.9 | 38.2 | 38.2 | 38.0 | 38.4 |
| G2 | 15.6 | 21.8 | 24.0 | 23.8 | 26.2 | 30.2 | 18.7 | 19.5 | 19.1 | 19.1 | 19.0 | 19.2 |
| S | 16.1 | 22.1 | 24.3 | 24.0 | 26.1 | 33.0 | 19.6 | 21.6 | 20.5 | 20.6 | 20.2 | 21.1 |
| B | 9.2 | 12.2 | 13.5 | 13.7 | 14.8 | 17.1 | 15.6 | 16.9 | 16.4 | 16.4 | 16.3 | 16.5 |
| F | 93.2 | 95.2 | 96.0 | 96.0 | 96.5 | 97.5 | 95.6 | 97.6 | 97.3 | 97.2 | 97.2 | 97.3 |

DGP = derived glycan property expressed as % of the total area;
min = minimum;
Q1 = first quartile (25. Percentile);
MV = mean value;
MD = median;
Q3 = third quartile (75. Percentile);
max = maximum.

Variability was expressed as an interquartile range from the first to the third quartile. In the analyzed group of female subjects, there was no significant deviation in the level of derived IgG glycan properties in comparison to the control samples. The fucosylation and monogalactosylation of IgG glycans had the lowest variability within the examined cohort, while the most variable glycosylation property was connected with agalactosylated IgG glycans.

Menstrual Cycles of Examined Female Subjects

The information about the menstrual cycles of the examined female subjects was reported through a questionnaire at each blood sampling. Thus collected data were employed for the calculation of the menstrual cycle durations during the study. The description of the menstrual cycles of the examined female subjects during the study is presented in Table 7.

TABLE 7
Menstrual cycles of the examined female subjects included in
the study.

| | | | Selected for statistical |
|---|---|---|---|
| No. | Parameter | Analyzed | analysis |
| 1 | Number of female subjects | 70 | 60 (85.6%) |
| | (N) | | |
| 2 | Plasma samples (number) | 774 | 500 (65%) |
| 3 | Menstrual cycles (number) | 208 | 140 (67.3%) |
| 4 | Duration of menstrual cycle | 31.3 ± 6.8 | 30 ± 4 |
| | (days): | | |
| | minimal: | 20 | 26 |
| | maximal: | 72 | 34 |
| 5 | Menstrual cycles per | 3 | 2 |
| | subject (number) | | |
| 6 | Plasma samples per | 4 | 4 |
| | menstrual cycle (number) | | |

Although all included subjects stated that they had regular and normal menstruation cycles, our results led to certain deviations. The most profound aberrations were observed regarding the menstruation period duration. During the study, the shortest menstrual cycle was 20 days only, whilst the longest was 72 days.

Despite this huge difference in the duration of the menstrual cycles, performed analysis revealed that most of the women (86%) had normal menstrual cycles that took between 26 and 34 days with an average duration of 30 days; see literature reference 20. For statistical analysis, 140 normal menstrual cycles (about 70% of all monitored cycles) were selected, from 500 samples of plasma from 60 female subjects.

Approximation of Menstrual Cycle Model

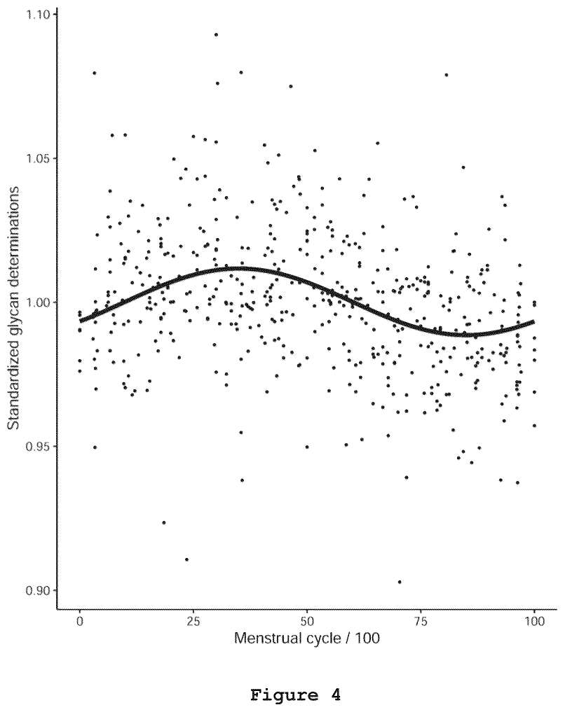

For the purpose of further statistical analysis, selected data from 140 menstrual cycles were grouped within the common model of menstrual cycle; see FIG. 4.

Concerning the fact that selected menstrual cycles had different duration times (from 26 to 34 days), they were normalized. The normalization was performed by dividing the duration of each menstrual cycle by 100%. In this manner, all glycan data and results for the sex hormone concentrations at various time points within each menstrual cycle could be simply positioned in the common menstrual cycle model, which enabled the required statistical analysis.

Variability of N-Glycosylation During the Menstrual Cycle

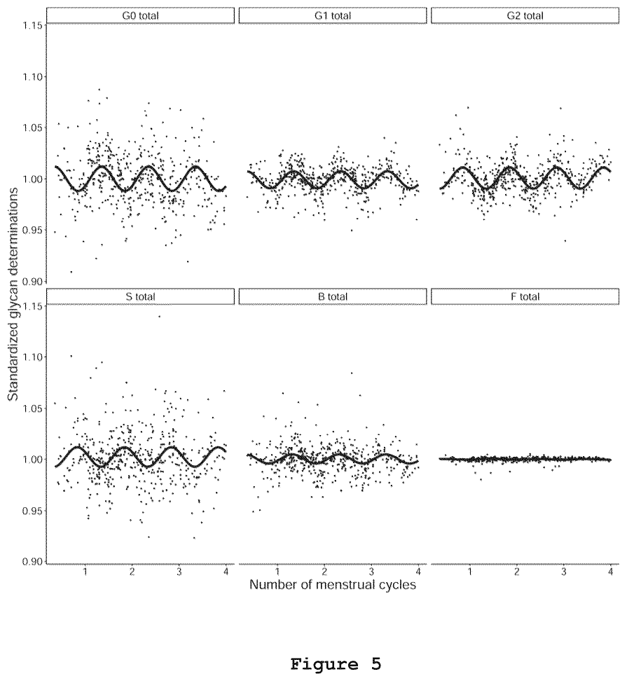

One of the main goals of this study was to determine whether the IgG glycan profile is changing with the fluctuation of sex hormones. The glycan data results from 140 selected menstrual cycles were analyzed in the said model menstrual cycle. The periodical and cyclic dynamic of the level of agalactosylated (G0), monogalactosylated (G1), digalactosylated (G2), and sialylated (S) glycans, as well as bisecting glycans (B) in total IgG N-glycome, was discovered by the analysis from longitudinal glycan measurements. The level of fucosylated glycans remained unchanged during the menstrual cycle; see FIG. 5.

The Relationship of IgG N-Glycosylation with Menstrual Cycle Phases

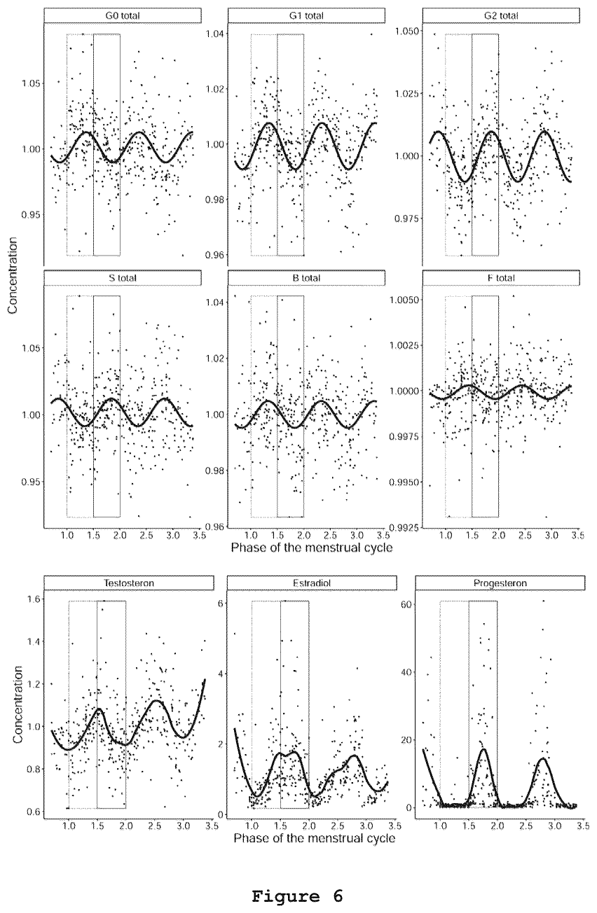

Since it was found that IgG N-glycosylation changes during the course of the menstrual cycle, it was necessary to determine the detailed specific changes in glycans composition. During the analysis of the glycosylation profile dynamic, two (2) patterns regarding the direction and the scope of glycan structure changes in particular stages of the menstrual cycle were found. The groups of digalactosylated (G2) and sialylated (S) glycans had the same change pattern and reached their highest level in the luteal phase of the menstrual cycle. On the other hand, the group of glycans consisting of agalactosylated (G0) and monogalactosylated (G1) glycans, as well as bisecting GlcNAc glycans had their own pattern of changes, which reached its highest level in the follicular phase of the menstrual cycle; see FIG. 6.

The Relationship of IgG N-Glycosylation and Sex Hormones During the Course of the Menstrual Cycle

Due to the fact that certain specific changes in glycan structures always occur during specific phases of the menstrual cycle, it was necessary to determine whether these changes are connected with the change in sex hormones concentrations during the course of the menstrual cycle. Despite the fact that there exist some studies that suggest some connection between the effect of sex hormones, especially estrogens, on IgG sialylation and galactosylation, the comparison of sex hormones dynamic pattern and IgG glycan properties, it was discovered that the highest concentration of estradiol (E2) does not correspond to the point when the presence of digalactosylated (G2) and sialylated (S) glycans is in their highest level; for comparison, see literature references 17 and 18:
17) C. Engdahl, A. Bondt, U. Harre, J. Raufer, R. Pfeifle, A. Camponeschi, M. Wuhrer, M. Seeling, I. L. MArtensson, F. Nimmerjahn, G. Krönke, H. U. Scherer, H. Forsblad-d'Elia, G. Schett: Estrogen induces St6gall expression and increases IgG sialylation in mice and patients with rheumatoid arthritis: a potential explanation for the increased risk of rheumatoid arthritis in postmenopausal women, Arthritis Res. Ther. 20 (2018) 84.
18) A. Ercan, W. M. Kohrt, J. Cui, K. D. Deane, M. Pezer, E. W. Yu, J. S. Hausmann, H. Campbell, U. B. Kaiser, P. M. Rudd, G. Lauc, J. F. Wilson, J. S. Finkelstein, P. A. Nigrovic: Estrogens regulate glycosylation of IgG in women and men. JCI Insight 2 (2017) e89703. doi: 10.1172/jci.insight.89703.

The day of the menstrual cycle, when the highest levels (peaks) of IgG glycan properties and sex hormones were observed is shown in Table 8. The highest level of digalactosylated (G2) and sialylated (S) IgG glycans is approximately 25. day of the luteal phase, what is a 12-days shift from the highest estradiol (E2) concentration at approximately 13. day of the follicular phase of the menstrual cycle. Furthermore, the highest level of digalactosylated (G2) and sialylated (S) IgG occurs 9 days after the highest testosterone (T) concentration which is approximately at 16. day of the menstrual cycle and simultaneously with the highest progesterone concentration, in the luteal phase of the menstrual cycle; see FIG. 6.

TABLE 8
The highest values (peaks) of derived IgG N-glycan
properties and sex hormones levels within the menstrual cycle.

| Sex | Peak | Day | GPR | Peak | Day | Time shift (days) | | |
|---|---|---|---|---|---|---|---|---|
| hormone | (% MC) | MC | IgG | (% MC) | MC | T > GPR | E2 > GPR | P > GPR |
| T | 55 | 16. | G0 | 35 | 10. | 24 | 27 | 15 |
| E2 | 45 | 13. | G1 | 35 | 10. | | | |
| P | 82 | 25. | G2 | 85 | 25. | 9 | 12 | ist |
| | | | S | 84 | 25. | | | |
| | | | B | 31 | 9. | 23 | 26 | 14 |
| | | | F | 47 | 14. | ist | ist | 29 |

MC = menstrual cycle;
GPR = glycan property: G0, G1, G2, S, B, F;
T = testosterone;
E2 = estradiol;
P = progesterone;
ist = in the same time.

The largest representation of agalactosylated (G0), monogalactosylated (G1), and bisecting GlcNAc on IgG did not match with any of the points with the highest sex hormones concentration during the menstrual cycle. Instead, the levels of bisecting GlcNAc on IgG reached their maximum at approximately 9. day of the menstrual cycle and the levels of agalactosylated (G0) and monogalactosylated (G1) IgG were the highest at 10. day of the follicular phase of the menstrual cycle. This happens after the luteal-follicular phase, which is a transitional one in between two menstrual cycles, and which is generally connected with the lowest levels of monitored sex hormones; see literature reference 19:
19) B. G. Reed, B. R. Carr: The Normal Menstrual Cycle and the Control of Ovulation. 2018 Aug. 5. In: K. R. Feingold, B. Anawalt, A. Boyce, G. Chrousos, W. W. de Herder, K. Dungan, A. Grossman, J. M. Hershman, J. Hofland, G. Kaltsas, C. Koch, P. Kopp, M. Korbonits, R. McLachlan, J. E. Morley, M. New, J. Purnell, F. Singer, C. A. Stratakis, D. L. Trence, D. P. Wilson (editori). Endotext [Internet]. South Dartmouth (MA): MDText.com, Inc.; 2000. PMID: 25905282.

The day of the menstrual cycle in which the highest average value of said parameters and their mutual time shift was calculated according to equations (1) and (2). Since there was discovered a discrepancy between the dynamic of sex hormones and IgG glycan properties within the menstrual cycle, it was necessary to study the eventual relationship between these time-shifted events. The analysis of the relationship between sex hormones and IgG glycan properties within the menstrual cycle is presented in Table 9.

TABLE 9
The relationship between sex hormones concentration and the
dynamic of IgG N-glycome changes within the menstrual cycle.

| | | | | Hormone | | |
|---|---|---|---|---|---|---|
| | | | Hormone | effect on | | |
| | | Peak GPR | peak | the level | | Corr. |
| GPR | Hormone | (% MC) | (% MC) | of GS | p-value | p-value |
| G0 | T | 35 | 55 | positive | 0.002 | 0.003 |
| | E2 | 35 | 45 | positive | 0.211 | 0.253 |
| | P | 35 | 82 | negative | [symbol] | [symbol] |
| G1 | T | 35 | 55 | positive | 0.001 | 0.002 |
| | E2 | 35 | 45 | negative | 0.001 | 0.002 |
| | P | 35 | 82 | negative | [symbol] | [symbol] |
| G2 | T | 85 | 55 | negative | [symbol] | [symbol] |
| | E2 | 85 | 45 | positive | 0.074 | 0.095 |
| | P | 85 | 82 | positive | [symbol] | [symbol] |
| S | T | 84 | 55 | negative | 0.016 | 0.024 |
| | E2 | 84 | 45 | positive | 0.032 | 0.045 |
| | P | 84 | 82 | positive | [symbol] | 0.001 |
| B | T | 31 | 55 | positive | 0.529 | 0.56 |
| | E2 | 31 | 45 | negative | 0.002 | 0.004 |
| | P | 31 | 82 | negative | [symbol] | [symbol] |
| | T | 47 | 55 | positive | 0.013 | 0.021 |
| | E2 | 47 | 45 | positive | 0.285 | 0.32 |
| | P | 47 | 82 | negative | 0.784 | 0.784 |

GPR = derived glycan property: agalactosylated glycans (G0), monogalactosylated glycans (G1), digalactosylated glycans (G2), sialylated glycans (S), bisecting glycans based on GlcNAc (B), core-fucosylated glycans (F);
MC = menstrual cycle;
T = testosterone;
E2 = estradiol;
P = progesterone;
Peak GPR = a time point within the menstrual cycle, expressed in percentage (%) in which the highest level of IgG glycans of similar structural properties was observed;
Hormone peak = a time point within the menstrual cycle, expressed in percentage (%), in which the highest concentration of analyzed sex hormone was detected;
p-value = describes the statistical significance of the functional effects of the respective hormone on each particular glycan structural property within the menstrual cycle;
Corr. p-value = corrected (adjusted) p-values on multiple tests according to the Benjamini-Hochberg method. Statistically significant values are those where said p-values are lower than 0.05 (marked in bold). The duration of one menstrual cycle is 100%. The follicular phase is 0% to 50%, while the luteal phase is from 50% to 100% of the menstrual cycle.

The results show that all structural properties that described the IgG glycosylation pathways have statistically significant associations with the concentration of estradiol (E2), progesterone (P), and testosterone (T) in the menstrual cycle. Furthermore, the analysis showed that progesterone (P) and estradiol (E2) have the same direction of functional effects on IgG glycosylation patterns. This suggests that estradiol (E2) is positively connected with sialylation, while progesterone (P) is with both sialylation and digalactosylation of IgG within the menstrual cycle. Additionally, estradiol (E2) and progesterone (P) are negatively connected with the production of IgG glycoforms containing bisecting GlcNAc (B) or monogalactosylated (G1) glycans, whilst P is negatively connected with agalactosylation (G0).

On the other hand, testosterone (T) exhibits the opposite effect on IgG glycosylation, yielding negative functional effects on digalactosylation (G2), sialylation (S), and positive effects on agalactosylated (G0), monogalactosylated (G1), and fucosylated (F) IgG glycans within the menstrual cycle.

The Quantitative Influence of the Menstrual Cycle on IgG Glycosylation

The scope of IgG glycome changes during the course of the menstrual cycle is presented in Table 10.

TABLE 10
The relationship of menstrual cycle and variability of IgG
N-glycosylation.

| | Peak | Variability | | | | | |
|---|---|---|---|---|---|---|---|
| | GPR | GPR | | Person | MCVar | | Corr.p |
| GPR | (% MC) | (%) | SD | Var | (%) | P-values | values |
| G0 | 35 | 1.1 | 0.05 | 98 | 0.12 | [symbol] | [symbol] |
| G1 | 35 | 0.8 | 0.12 | 92 | 0.72 | [symbol] | [symbol] |
| G2 | 85 | 1.0 | 0.08 | 99 | 0.30 | [symbol] | [symbol] |
| S | 84 | 1.0 | 0.07 | 90 | 0.26 | [symbol] | [symbol] |
| B | 31 | 0.5 | 0.04 | 86 | 0.06 | 0.01 | 0.01 |
| F | 47 | 0.5 | 0.05 | 93 | 0.10 | 0.09 | 0.09 |

GPR = derived glycan property: agalactosylated glycans (G0), monogalactosylated glycans (G1), digalactosylated glycans (G2), sialylated glycans (S), bisecting glycans based on GlcNAc (B), core-fucosylated glycans (F);
MC = menstrual cycle;
SD = standard deviation;
Peak GPR = a time point within the menstrual cycle, expressed in percentage (%), in which the highest level of IgG glycans of similar structural properties was observed;
Variability GPR = an effect of each particular menstrual cycle phase upon the derived glycan property, expressed in percentage (%) and corresponding standard deviation (SD)—it was calculated from the ratio of average values of the highest level (peaks) and all measurements of glycan property in particular phase of the menstrual cycle;
PersonVar is the variability of IgG glycan property that originates from the differences in IgG glycosylation between different female subjects;
MCVar is the variability of IgG glycan property that originates due to the menstrual cycle;
P-value = describes the statistical significance of the functional effects of the respective hormone on each particular glycan structural property within the menstrual cycle;
Corr. p-value = corrected (adjusted) p-values on multiple tests according to the Benjamini-Hochberg method. Statistically significant values are those where said p-values are lower than 0.05 (marked in bold) . The duration of one menstrual cycle is 100%. The follicular phase = 0% to 50%, while the luteal phase is from 50% to 100% of the menstrual cycle.

The variability scope of the IgG glycan properties connected with the phase of the menstrual cycle is very small, from 0.5-1.1%. The variation of galactosylated and sialylated glycans, which changed the most during the menstrual cycle, was 1.1% (agalactosylated; G0), 1.0% (sialylated; S). Moderately changeable were monogalactosylated (G1) at 0.8% and bisecting (B) at 0.5%. The levels of fucosylated glycans were not changed during the menstrual cycle.

Furthermore, it was determined to what extent the menstrual cycle itself contributes to the IgG N-glycosylation variability. Analysis showed that the menstrual cycle could explain 0.06% variability for bisecting GlcNAc (p=0.01) to maximally 0.72% with monogalactosylated (G1) glycans (p=3.36·10⁻²²). For instance, the difference in the pattern of how each particular female subject underwent IgG glycosylation explains 86% of variations at bisecting GlcNAc, while the level of agalactosylation between two (2) subjects could differ for even 98%. Therefore, the results reveal that the changes in IgG N-glycome, which are caused by the menstrual cycle itself, represent less than 0.8% of the variability at the level of any studied glycan property, within the studied cohort of female subjects.

Other aspects of the analysis of the study results are described in Example 2.

Statistical Processing of the Results from the Study and the Formation of a Numerical Model for Determining Average Multiday Estradiol (E2) Concentration and Perimenopause and Menopause Diagnosis

To be able to compare the areas under the chromatographic peak of chromatograms of different samples, relative areas under the peaks were calculated by dividing the area under the respective peak with total area of the corresponding chromatogram. The generated relative areas were logit transformed according to equation (3). This enables the approximation of relative area distribution with normal distribution. The influence of the series on measurement was eliminated by the use of ComBat method (R package “Surrogate Variable Analysis”); see literature reference 20:

$$log it ( x ) = ln ( \frac{x}{1 - x} ) ; \qquad ( 3 )$$

$$x ∈ 〈 0.1 〉$$

20) J. T. Leek, W. E. Johnson, H. S. Parker, A. E. Jaffe, J. D. Storey: The sva package for removing batch effects and other unwanted variation in high-throughput experiments, Bioinformatics 28 (2012) 882-883; doi:10.1093/bioinformatics/bts034.

These data were used in all further analyses. For determination of the relationship between menopause and IgG N-glycome, a linear mixed model (R package “lme4”) was used; see literature reference 21:
21) D. Bates, M. Machler, B. Bolker, S. Walker: Fitting Linear Mixed-Effects Models Using lme4, J. Stat. Softw. 67 (2015) doi:10.18637/jss.v067.i01.

For each glycan structure, the model was adjusted by the way that the dependent variable was logit transformed relative area of the respective glycan. The fixed factors were the menopause status and the age. The latter was included in the menopause status factor in order to estimate the influence of the age of the examined female on glycans change (depending on the menopause status). The dependence of particular measurements, as a consequence of the study design in which some subjects were sampled one to three times and which belonged to the same family (twins), was controlled by the random effects. The latter were unique subject code, included in unique code for the family as random sections and age as a random bias. Thus estimated average relative area, corrected on the age effects (and the corresponding 95% confidence interval), was determined by the use of an adjusted model for samples of women before and after menopause onset. The estimated values were compared with a post-hoc t-test with adjustment for multiple testing against the Benjamini-Hichberg method (R package “emmeans”); see literature references 22 and 23:
22) R. Lenth: Emmeans: Estimated Marginal means, aka Least-Squares Means. R Package Version 1.5.4; vidjeti poveznicu: https://cran.r-project.org/web/packages/emmeans/index.html;
23) Y. Benjamini, Y. Hochberg: Controlling the False Discovery Rate: A Practical and Powerful Approach to Multiple Testing, J. R. Stat. Soc. Ser. B 57 (1995) 289-300. doi:10.1111/j.2517-6161.1995.tb02031.x.

Furthermore, average annual changes in relative areas (and corresponding 95% confidence interval) for samples taken from women before and after menopause onset were estimated. The comparisons were also made by the post-hoc t-test with adjustment for multiple testing according to the Benjamini-Hochberg method, described in literature references 22 and 23.

The obtained results of estimated average values are graphically presented with a dot, and the corresponding 95% confidence interval is shown as an error bar. The statistical significance of the difference between the values obtained before and after the menopause onset was shown on the graphical display as the p-value (corrected for multiple testing) above shown average values (R package “ggplot 2”); see literature reference 24:
24) H. Wickham: ggplot2: Elegant Graphics for Data Analysis (2016) Springer-Verlag, New York, SAD.

The Development of the Numerical Model for the Determination of Multiday Average Concentration of Estradiol (E2)

Model A: A model based on the N-glycome of a single sample. Data from analyzed samples were divided into subgroup for training the model and on subgroup for model testing. The subgroup for model training is based on randomly selected measurements from each family in order to eliminate mutual dependence between samples. The testing subgroup contained all remaining data. The L1-regulated logistic model was adjusted, which as a dependable variable had the menopause status (dichotomous variable —“yes” or “no”), while as independent variables, logit transformed [equation (3)] relative areas under all peaks of all N-glycans were taken; see literature reference 25:
25) J. Friedman, T. Hastie, R. Tibshirani: Regularization Paths for Generalized Linear Models via Coordinate Descent, J. Stat. Softw. 33 (2010) 1-22; doi:10.18637/jss.v033.i01.

L1-regularization, known as Lasso regularization, was employed in order to prevent overtraining and decrease the complexity of the final model. The method of ten-fold cross-checking was used for the calculation of independent variable on the subgroup for the training model. Hyperparameter λ=4.5×10⁻² is employed for decreasing predictors number to 4 or less (R package “caret”); see literature reference 26:
26) M. Kuhn: caret: Classification and Regression Training (2020). R package version 6.0-86. https://CRAN.R-project.org/package=caret.

The probability that IgG N-glycome comes from the women population that passed perimenopause and entered menopause was calculated by using the formula of the final model. The obtained menopause predictions and real menopause statuses were analyzed with ROC (Receiver Operating Characteristic) analysis (R package “pROC”), and these results are shown with the ROC curve; see literature references 24 and 27:
27) X. Robin, N. Turck, A. Hainard, N. Tiberti, F. Lisacek, J.-C. Sanchez, M. Müller: pROC: an open-source package for R and S+ to analyze and compare ROC curves. BMC Bioinform. 12 (2011) 77. doi:10.1186/1471-2105-12-77.

The confidence interval (at 95%) of the area under ROC curve was determined by the method of repeated sampling (bootstrap) with 2,000 samples. To demonstrate that the whole IgG N-glycome is responsible for menopause status prediction and not particular glycans, the complete procedure of this section was repeated two times more, with the elimination of glycans as predictors that were selected in the final model from earlier iterations.

Model B: This model is based on average annual IgG N-glycome changes. Average annual changes were calculated by dividing the difference of logit transformed relative areas under the peak with a difference in age between the sample points expressed in years, according to equation (4):

$$dGPB = \frac{log it ( {GPB}_{2} ) - log it ( {GPB}_{1} )}{age 2 - age 1} ; \qquad ( 4 )$$

$$age 2 > age 1$$

dGPB—average annual difference; GP—relative area under the peak which corresponds to particular glycan GPB1-GPB24; age—age in years; indexes 1 and 2 represents the sampling time

The combination of model A and model B: The development of the model which combines the information on average annual changes in IgG N-glycome and the levels of IgG N-glycome structures caused in the second time point, was adjusted to the same subgroup of data as the model based only on average annual change of IgG N-glycome.

The testing subgroup was equal to the testing subgroup based only on average annual changes in IgG N-glycome. For this purpose, an adjusted L1-regularized logistic model was employed, which used the menopause status as a dependent variable (dichotomic variable—“yes or “no”), while average annual changes were taken as an independent variable, according to equation (4) and relative areas under all peaks of IgG N-glycome were logit transformed by the equation (3). Relative areas under all IgG N-glycome chromatographic peaks were sampled in the second time point; see literature reference 25. The coefficients of independent variables of the model were calculated by the use of the method of ten-fold cross-checking. The hyperparameter 2=0.1 was used for the decreasing of predictors number to 5 or less (R package “caret”); see literature reference 26.

The probability that measured N-glycome comes from the women population who passed perimenopause and entered menopause was calculated with the final model formula. Obtained results for the menopause prediction and the real menopause statuses were analyzed by ROC (Receiver Operating Characteristic) analysis (R package “pROC”), and the results were presented by ROC curve; see literature references 24 and 27. The confidence interval (95% level) of the area under the ROC curve was determined by the bootstrap method with the samples number 2,000. To demonstrate whether the whole IgG N-glycome is responsible for the estradiol (E2) concentration predictivity, and not the levels of particular glycans, the complete procedure was repeated two times more. This resulted in the selection of glycans important for the final model of previous iterations.

The final numerical model for the calculation of the multiday average concentration of estradiol (E2) from the blood of examined female subjects, and from the results of quantitative IgG N-glycan analysis, is as follows:

Log c(E2)=−15.529·GPB4−2602·GPB8+5.589·GPB10+9.699·GPB12+53.911·GPB15+9.901·GPB16−1.990·GPB2·GPB10−0.065·GPB2·GPB12+3.601·GPB2·GPB15+0.007·GPB2·GPB16+0.465·(GPB4)^{2+2.889}·GPB4·GPB8+5.106·GPB4·GPB10−0.817·GPB4·GPB12−8.606·GPB4·GPB15+1.490·GPB4·GPB18+1.689·(GPB8)²−9.048·GPB8·GPB10−0.999·GPB8·GPB12−2.253·GPB8·GPB15+3.143·(GPB10)²+0.712·GPB10·GPB12−3.505·GPB10·GPB15−4.753·GPB10·GPB16+1.128·GPB10·GPB18−4.584·GPB12·GPB15+1.138·GPB12·GPB16−1.355·GPB12·GPB18−0.598·GPB12·GPB22−0.904·GPB12·GPB23−4.638·(GPB15)²+0.287·GPB15·GPB16−3.049·GPB15·GPB18+2.492·(GPB16)²−3.041·GPB16·GPB18

wherein factors GPB2, GPB4, GPB8, GPB10, GPB12, GPB15, GPB16, GPB18, GPB22 and GPB23 represent natural logarithms of corresponding values belonging to relative areas under the peaks of the respective glycans GPB2, GPB4, GPB8, GPB10, GPB12, GPB15, GPB16, GPB18, GPB22, GPB23 obtained from the chromatogram, given by the selected quantitative analytical technique, and from which the multiday average molar concentration of estradiol c(E2) is calculated and expressed in picomoles per liter (pmol/L).

The Use of the Method According to the Present Invention and Corresponding Numerical Model in Clinical Practice

The diagnostic process according to the present invention is used for the determination of the average multiday molar concentration of estradiol (E2) in the blood for a 3-month period, preferably a 2-month period and most preferably a 1-month period.

Additionally, the diagnostic process from the present invention is used for the determination of whether the examined female subject has passed through perimenopause and entered into menopause.

Alternatively, the process is used for the determination whether the examined female subject has entered into perimenopause.

### Examples

General Remarks

The nomenclature of IgG N-glycans, e.g., FA1, A2, A2B, etc., is derived according to the rules of the Oxford nomenclature.

The meaning of the abbreviations used is as follows:
2AB=2-aminobenzamide;
CMIA=chemiluminescent microparticle immuno assay
DMF=N,N-dimethylformamide, a solvent;
DMSO=dimethyl sulfoxide, a solvent;
e=Euler's number;
EDTA=N,N,N′,N′-ethylenediamine-tetraacetic acid, disodium salt dihydrate;
FLR=fluorescence (detector for UPLC instrument)
HILIC=hydrophilic interaction liquid chromatography;
IgG=immunoglobulin G;
MC=menstrual cycle;
2PB=2-picoline borane
PBS=phosphate-buffered saline, a buffer solution
PNGase F=enzyme peptide-N4-(N-acetyl-beta-glucosaminyl)asparagine amidase F;
PR=procainamide;
RF=5-dioxopyrrolidine-1-yl-[2N-(2-(N′,N′-diethylamino)ethyl)carbamoyl]-quinoline-6-yl-carbamate (RapiFluor-MS);
r.t.=room temperature
RT=retention time (t_{R}); of corresponding glycans in analytical, e.g., UPLC method;
SD=standard deviation;
SDS=sodium dodecylsulfate, a surfactant;
Tris=tris(hydroxymethyl)aminometane, a buffer;
UPLC=ultra-high performance liquid chromatography.

Chemicals, reagents, and accessories used in this research are purchased from the following suppliers: 2-aminobenzamide (2AB): Sigma-Aldrich (US); 2-picoline borane (2PB): Sigma-Aldrich (US); acetonitrile, HPLC grade: Scharlab; acetonitrile LC-MS grade: J. T. Baker (US); ammonium formate (HCOONH₄): Acros Organics (BE); dimethyl sulfoxide (DMSO): Sigma-Aldrich (US); ethanol: Carlo Erba (IT); formic acid (HCOOH): Merck (DE); Igepal CA-630: Sigma-Aldrich (US); potassium dihydrogen phosphate (KH₂PO₄): Sigma-Aldrich (US); potassium chloride (KCl): EMD Millipore (US); hydrochloric acid (HCl): Kemika (HR); sodium dodecylsulfate (SDS): Sigma-Aldrich (US); sodium hydrogen phosphate (Na₂HPO₄): Acros Organics (BE); sodium hydrogen carbonate (NaHCO₃): Merck (DE); sodium hydroxide (NaOH): Kemika (HR); sodium chloride (NaCl): Carlo Erba (IT); acetic acid (CH₃COOH): Merck (DE); ammonia solution: Merck (DE); tris (hydroxymethyl) aminometane: Acros Organics (BE); ultrapure water: Millipore (US); PNGase F (10 U/mL): Promega; ARC Estradiol RGT: Abbot Diagnostics (US); ARC Progesterone RGT: Abbott Diagnostics (US); ARC 2^{nd} Gen Testo RGT: Abbott Diagnostics (US); ARC Trigger solution: Abbott Diagnostics (US); ARC Pre-trigger solution: Abbott Diagnostics (US); GHP Acroprep 0.20 μm filter plate: Pall Corp. (US); GHP Acroprep 0.45 μm filter plocica: Pall Corp. (US); Supor PES filtar: Nalgene (US); plate for samples collection with 96-wells, 1-2 mL volume: Waters (US); Protein G plate: BIA Separations (SI); monolithic plate with protein G (96-wells): BIA Separations (SI).

The research was performed by the use of the following instruments: ARCHITECT® i1000SR analyser: Abbott Diagnostics (US); ABgene PCR plates: Thermo Scientific (US); Acquity UPLC Glycan BEH amide column, 130 Λ, 1.7 μm, 2.1 mm×100 mm: Waters (US); Acquity UPLC H-Class system: Waters (US); reaction tubes ARC: Abbott Diagnostics (US); centrifuge, model 5840: Eppendorf (DE); Fume cupboard DIGIM 15 AFM: Schneider (FR); Water purification system Direct-Q 3UV: Millipore (US); analytical balance Explorer©: Ohaus Corporation (US); pH-meter FiveEasy™: Mettler Toledo (CH); precise balance JL1502-G: Mettler Toledo (CH); laboratory oven LAB. HOT AIR OVEN, M.R.C.; laboratory incubator: M.R.C.; centrifuge miniSpin: Eppendorf (DE); magnetic stirrer MR 3000 D: Heildoph (DE); spectrophotometer Nanodrop ND-8000: Thermo Scientific (US); Pipet-Lite XLS manual micropipette Rainin: Mettler Toledo (CH); circular shaker, model 3023: GFL; vacuum concentrator Savant SC210A SpeedVac and Savant solvent trap: Thermo Scientific (US); Refrigerated Vapor Traps RVT400 and vacuum pump OFP400: Thermo Scientific (US); vacuum manifold and vacuum pump: Pall (US); laboratory shaker Vortex-Genie 2: Scientific Industries (US).

The isolations of blood plasma samples from female subjects were performed by the methodology known in the prior art; see literature reference 4.

### Example 1. Isolation of Immunoglobulin G (IgG) from Human Plasma, Rapid Deglycosylation of IgG, Glycans Purification, Fluorescent Derivatisation of Glycans with 2-Aminobenzamide (2AB) and the Method for Quantitative Analysis of Thus Released and Labeled Glycans

The isolation of IgG from blood plasma was conducted by the common process known in the prior art; see literature references 3 and 11.

Isolation of IgG

IgG was isolated from the blood plasma samples by affinity chromatography by binding to a 96-wells protein G plate with a vacuum device for the plate filtration. All steps of IgG isolation were carried out at 380 mmHg pressure, while at the application of plasma samples and IgG elution, the reduced pressure at around 200 mmHg was employed. The solutions used for the isolation were previously filtered through a 0.2 μm filter (Supor PES filter). Before the application of the plasma samples, the protein G plate was washed with 2 mL ultrapure water (18 MΩ/cm at 25° C.), 2 mL concentrated PBS buffer, pH=7.4 (137 mmol/L NaCl, 2.7 mmol/L Na₂HPO₄, 9.7 mmol/L KH₂PO₄, 2.2 mmol/L KCl; titrated with NaOH to pH=7.4), 1 mL 0.1 mol/L formic acid, pH=2.5; 2 mL 10× concentrated PBS buffer, pH=6.6; and adjusted with 4 mL 1× concentrated PBS buffer, pH=7.4. Except sample from female subjects (100 μL), per five aliquots of standard plasma samples (50 μL) were randomly added in each plate, while per one well was left empty as a negative control. The plasma samples were mixed and centrifuged at 1,479 g for 10 minutes. Then, the samples were diluted by the addition of 1× concentrated PBS buffer, pH=7.4 in ratio 1:7, V/V, and filtered through 0.45 μm GHP AcroPrep filter plate with 96 wells, by the use of vacuum device for plates (Pall). Filtered plasma samples were applied on protein G plate and washed 3×2 mL 1× concentrated PBS buffer, pH=7.4 to remove unbounded proteins. The bounded IgG was eluted from the protein G plate with 1 mL 0.1 mol/L formic acid and neutralized with 170 μL 1 mol/L ammonium hydrogencarbonate. The remained protein G plate was regenerated for repeated use by washing with 1 mL 0.1 mol/L formic acid, 2 mL 10× concentrated PBS buffer, pH=6.6, 4 mL 1× concentrated PBS buffer, pH=7.4 and 1 mL buffer for storage of protein G plate (ethanol, a=20%; 20 mmol/L tris; 0.1 mol/L NaCl; titrated with HCl up to pH=7.4) and additional 1 mL of the buffer for storage was added and stored at +4° C.

Determination of IgG Concentration

The IgG concentration was determined by measuring absorbance at 280 nm with a Nanodrop ND-8000 spectrophotometer (Thermo Scientific; US). A part of IgG eluate was separated and dried in rotary vacuum concentrator SpeedVac Concentrator SC210A (Thermo Scientific; US). The prepared samples were stored at −20° C. till further use.

The Denaturation and Deglycosylation of IgG in Solution

Dried IgG samples were denatured with 30 μL SDS (γ=1.33%) and incubated at 65° C. for 10 minutes. 10 μL of Igepal CA-630 solution (γ=4%) was added to each sample to deactivate the SDS excess. The plates are incubated at r.t. for 15 minutes. IgG molecules were deglycosylated by the addition of 10 μL 5× concentrated PBS buffer and 1.25 U PNGase F. The deglycosylation reaction was conducted at 37° C. for 18 h.

Fluorescent Labeling with 2-Amino-Benzamide (2AB) and Purification of 2AB-Derivatised IgG N-Glycans

Due to the fact that glycans do not contain chromophores, their content cannot be measured by any spectrophotometric technique. This is the reason why free N-glycans are derivatized with fluorescent reagents such as 2AB. The derivatization reaction was carried out with 2AB (γ=19.2 mg/mL) and 2-picoline borane (2PB; γ=44.8 mg/mL) dissolved in the solution of acetic acid (HOAc) and DMSO in ratio 30:70, V/V. To each sample, per 25 μL of said labelling solution was added and the samples were incubated at 65° C. for 2 h. After the derivatization reaction, all impurities were removed by solid phase extraction (HILIC-SPE). After cooling to r.t. for 30 minutes, to each sample, 700 PL acetonitrile (p=100%, 4° C.) was added and the samples were transferred to GHP AcroPrep 0.2 μm filter plate. The filter plate was previously washed with 200 μL ethanol (p=70%), 200 μL ultrapure water and cooled acetonitrile (p=96%, 4° C.). Between each of these steps, the filter plate was emptied with vacuum manifold and vacuum was not higher than 2 inHg. After transferring to GHP AcroPrep 0.2 μm filter plate, the samples were incubated at r.t. for 2 minutes. During this period, the binding of 2AB-labelled N-glycans to polypropylene membrane plate occurred. Each sample was then washed 4× with 200 μL cooled acetonitrile (p=96%, 4° C.) and then the glycans were eluted from membrane plate. The elution was conducted in two subsequent equal steps: to each well, 90 μL of ultrapure water was added followed by incubation at r.t. for 15 minutes with shaking on a circular shaker. Collected eluates were centrifuged at 164 g for 5 minutes into ABgene PCR plates. Purified fluorescently labeled IgG N-glycans were stored at −20° C. till further use.

Analysis of IgG Glycans by the UPLC Method

Labeled IgG N-glycans were analyzed by HILIC-UPLC method on amide ACQUITY UPLC© Glycan BEH column (Waters; US) of 100 mm length, diameter 2.1 mm and particles size 1.7 μm according to the method described in literature reference 3. The analyses were conducted on Acquity UPLC H-Class (Waters; US) instrument equipped with quaternary solvent manager QSM, sample manager SM) and fluorescent (FLR) detector. Instrument was controlled by program Empower 3, version 3471 (Waters; US).

The glycan samples were prepared by mixing with acetonitrile (p=100%) in ratio: sample:acetonitrile=20:80, V/V. As the mobile phase, ammonium formate, c=0.1 mol/L, pH=4.4 was used as solvent A and acetonitrile (p=100%) as solvent B. Between analyses, the system was washed with aqueous acetonitrile (p=75%). Samples were cooled to 10° C. before injecting, while the separation was carried out at 60° C. The analytical method uses a linear gradient with 25-38% solvent A at a flow rate of 0.4 ml/min, with 27 minutes run. Separated glycans were detected by FLR detector at wavelength for 2AB: λₑₓ=250 nm, λₑₘ=428 nm). The system was calibrated with fluorescently labeled glucose oligomers as an external standard.

The typical chromatogram obtained by this method is presented in FIG. 1, while the retention times (t_{R}) of thus separated IgG glycans GPB1-GPB24 are given in Table 2.

### Example 2. The Study of the Variability of IgG N-Glycans Against the Concentration of Sex Hormones and, Subordinate, Estradiol (E2) During Different Phases of the Menstrual Cycle in Female Subjects of 18-50 Years and the Development of the Numerical Model for the Calculation of Multiday Average Estradiol (E2) Concentration According to the Present Invention

All details of this study are described in the section Detailed Description of the Disclosure, including all experimental data, statistical analysis of the results, and all generated variants of the numerical model for the calculation of the multiday average concentration of estradiol (E2), from one or more blood analyses of examined female subjects, according to the present invention.

Further Details of this Study are as Follows:

As described earlier, the study was performed on 70 healthy female subjects from 18-50 years of age; see FIG. 2. All subjects signed their written informed consent for participation in the study, which was approved by the ethical committee of the Medical Faculty of Zagreb University, Croatia, and Medicinal Faculty of Beijing University, China. The study was carried out according to the principles of the Helsinki Declaration.

Study Protocol

For the purpose of IgG N-glycans analysis, the samples of blood plasma were collected. The samples of the blood, per 5 mL, by venipuncture to the tubes with EDTA anticoagulant (BD Vacutainer® K₂EDTA REF 368861 test tubes) by the routine procedure in Oil hospital Jidong, in Chinese province Hebei. The blood samples were incubated at r.t. for 30 minutes. Then the samples were centrifuged at 1,670 g for 10 minutes at 4° C. to separate the plasma. Isolated plasma samples were stored at −80° C. till further use.

Immunochemical Method for the Determination of Sex Hormones Concentration

The concentration of sex hormones estradiol (E2), progesterone (P), and testosterone (T) was determined in plasma samples by chemiluminescent microparticle immunoassay (CMIA) on ARCHITECT© i1000SR automatized instrument (Abbott Diagnostics; US) by using commercially available reagent sets for the determination of said hormones; see literature reference 14. Before measuring, the instrument had to be calibrated with calibration solutions for hormones whose concentration was determined. The reaction mixtures for the determination of sex hormones concentration were obtained by mixing the plasma samples, paramagnetic microparticles coated with antibodies to the respective hormone, and the conjugate of targeted hormone derived with acridine for disposable ARC reaction tubes (Abbott Diagnostics; US). To the microparticles coated with antibodies, the hormone from plasma was bound first, while the rest of the free antibodies on microparticles were bound on acridinylated hormone conjugate, which, in contact with pre-activating solution (H₂O₂, φ=1.32%) and activating solution (NaOH, c=0.35 M) generates the chemiluminescence reaction. The signal is detected by ARHITECT i System optics and expressed in relative light units (RLU). The hormone concentration in plasma is inversely proportional to detected RLU units. These analyses were performed in collaboration with the Endocrinology laboratory of the Hospital for Female Diseases and Childbirths, Clinical Hospital Zagreb, Croatia.

Other Aspects of the Study Results Analysis

Normalization and “batch”-correction of the data obtained after UHPLC analyses are carried out with the aim of removing experimental error. The normalization was performed by dividing the area of each particular chromatographic peak (glycan) by the total area of the respective chromatogram.

Before “batch”-correction, the normalized data were log-transformed due to the inclination of the data distribution to the right side (“right-skewness”). Log-transformed data were corrected to “batch” by using “ComBat” method (R package “sva”). In this manner, the technical sources of the variation were modeled as “batch” covariant; see literature reference 28:
28) J. T. Leek, W. E. Johanson, H. S. Parker, E. J. Fertig, A. E. Jaffe, Y. Zhang, J. D. Storey, L. C. Torres, Surrogate Variable Analysis. R package version 3.38.0 (2020).

Estimated “batch” effects were taken away from log-transformed measurements with the aim of experimental noise correction. The average estradiol (E2) value was calculated from each examined female subject on the basis of particular determination and the values are log-transformed. Before the development of the machine learning model, 33% of the data was separated to be used as a data set that will be employed for the final validation of results. The rest of the 67% data was used for the development of the linear model. Glycans, which will be included in the final model according to the present invention, were selected by the method for selection of the best subgroup by stepwise backward selection method. This was conducted with statsmodels module; see literature reference 29:
29) Seabold, Skipper, Josef Perktold: “statsmodels: Econometric and statistical modeling with python; Proceedings of the 9th Python in Science Conference (2010).

In this manner, ten (10) chromatographic peaks (glycans): GPB2, GPB4, GPB8, GPB10, GPB12, GPB15, GPB16, GPB18, GPB22 and GPB23 were selected, for which the p-value was <0.001. Furthermore, to improve the prediction of the average value for estradiol (E2) by the linear model, to said set of data, a polynomial combination of all glycans with the second degree (the class PolynomialFeatures) was added. The number of properties was reduced to 35 and the smallest number of properties was selected, after which R² was not significantly increased by using the class SelectKBest; as a selection function “mutual_info_regression” was employed. In such a manner, all data were transformed. To predict log-average values of estradiol (E2) based on values of the corresponding chromatographic peaks, the linear regression model was developed by the use of machine learning. Also, the parameters for the model were established by the use of the class GridSearchCV with cross-validation with the ShuffleSplit class. In this case, the data were divided ten times randomly in the fifth, of which one group of data was used for the validation of parameters. In this manner the following parameters were found as the best: ‘copy_X’: True, ‘fit intercept’: False, ‘normalize’: True. A model with said parameters had R²=0.551 on the test sample of the cross-validation performed by the previously described procedure. On the test sample (isolated before the model development) R²=0.547, the maximal error was 1.04 and the square of the mean error value was 0.09.

For the preparation of data and the model development by machine learning, the programming language Python version 3.7.6, Python package Scikit-learn, and Jupyter notebook, were employed; see literature references 30 and 31:
30) Scikit-learn: Machine Learning in Python, Pedregosa . . . JMLR 12 (2011) 2825-2830.
31) T. Kluyver, B. Ragan-Kelley, F. Perez: Jupyter Notebooks—a publishing format for reproducible computational workflows. In: F. Loizides, B. Schmidt (Ed.): Positioning and Power in Academic Publishing; Players, Agents and Agendas. Clifton, VA: IOS Press (2016) 87-90.

The perimenopause condition is generally characterized by significantly milder disturbances of normal sex hormones levels, which regulate the menstrual cycle. As a consequence, the milder spectrum of symptoms occurs in comparison to the full state of menopause; see literature references 6 and 7. Despite the fact that the numerical models from the present invention do not enable an accurate distinction between the perimenopause from the menopause status, it is clear to the person skilled in the art, that the level of IgG N-glycan changes as subsequent multiday estradiol (E2) concentration changes will be very probably milder in comparison to the full menopause phase. In this manner, the present diagnostic process obviously has certain predictive value even for the determination of perimenopause.

The present disclosure reveals the method for the determination of the average multiday molar concentration of estradiol (E2), and from this result, the estimation of perimenopause and menopause status in female subjects based on quantitative analysis of N-glycans bounded on immunoglobulin G (IgG). The said diagnostic method is applicable to female subjects of 40-55 years of age. It provides the possibility to determine whether the examined female subject has entered into the phase of menopause on the basis of one or more blood analyses.

Despite the fact that the numerical model from the present invention does not enable an accurate distinction the perimenopause from the menopause status, it is clear to the person skilled in the art, that the level of IgG N-glycan changes will be very probably milder in comparison to the full menopause phase. In this manner, the present diagnostic process obviously exhibits certain predictive value even for the determination of perimenopause.

Additionally, the revealed process enables the determination of the multiday average molar concentration of estradiol (E2), which is an important diagnostic parameter, hardly accessible by any known single diagnostic method applied to only one blood analysis.

### INDUSTRIAL APPLICABILITY

The present invention discloses a diagnostic process for the determination of multiday estradiol (E2) concentration and whether an examined female subject has entered the perimenopause or menopause phase, based on IgG N-glycan analysis from one or more blood samples. In this manner, the industrial applicability of the present invention is obvious.

1. A method for the assessment of perimenopause and menopause status in female subjects by an analysis of N-glycans (I), bound to immunoglobulin G <IgG>, 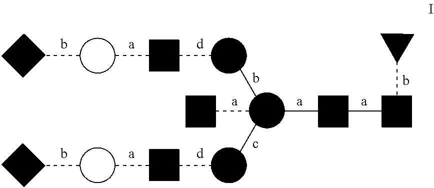
where the following symbols are used to denote chemical moieties:
▪ N-acetylglucosamine
▾ fucose
● mannose
◯ galactose
♦ N-acetylneuraminic acid
where letters a-d determine types of glycoside bond of N-glycans (I) respectively:
a=β<1-4>
b=α<1-6>
c=α<1-3>
d=β<1-2>
via determination of multiday average concentration of estradiol <E2> from this IgG N-glycans analysis: 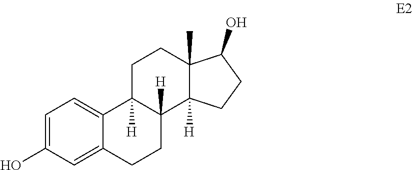
wherein the said method comprises the following steps:
a) isolation of plasma from one or more blood samples that have been collected from the female subject under examination,
b) release of said glycans from IgG,
c) quantitative analysis of thus released glycans in the free form or derivatized by fluorescent derivatization,
d) where the results from step c) are inserted in one or more numerical models suitable for the quantitative analysis used, where the said models are result of statistical data analysis performed in the study that determined the variation of quantitative IgG glycans content in the blood plasma of a female cohort that was not in the menstruation phase or any other known medical condition associated with sex hormones fluctuations, and where the selected model gives a numerical data indicative of the multiday average estradiol <E2> molar concentration in the blood of the examined female subject.

2. The process according to claim 1, wherein the female subject is between 40-55 years old.

3. The process according to claim 1, wherein the release of glycans I from IgG in the step b) is performed by chemical or enzymatic means with enzyme peptide-N4-(N-acetyl-beta-glucosaminyl)asparagine amidase F <PNGase F>.

4. The process according to claim 1, wherein the quantitative analysis in step c) is performed with ultra-performance liquid chromatography <UPLC>, MALDI-TOF mass spectrometry, coupled liquid chromatography, and mass spectrometry <LC-MS>, or capillary electrophoresis <CE>.

5. The process according to claim 1, wherein the set of glycans I, released from IgG, is fluorescently derivatized in the step c) with a combination of:
(a) an aromatic amine, and
(b) a reducing agent for reductive amination:
and the resulting mixture is analyzed by ultra-performance liquid chromatography <UPLC> for glycans GPB1-GPB 19 and GPB21-GPB24 defined below:

| No | Glycans | Code | Structure |
|---|---|---|---|
| 1 | F(6)A1 | GPB1 | 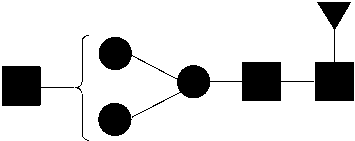 |
| 2 | A2 | GPB2 | 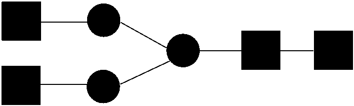 |
| 3 | A2B | GPB3 | 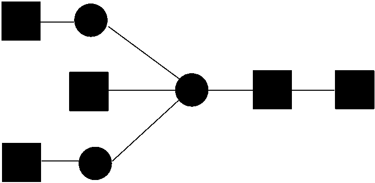 |
| 4 | F(6)A2 | GPB4 | 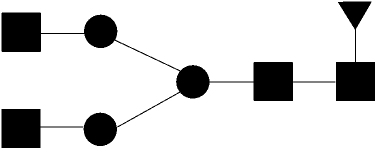 |
| 5 | M5 F(6)A2 | GPB5 | 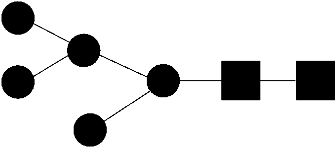 |
| | | | 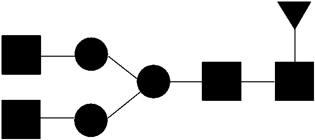 |
| 6 | F(6)A2B A2[6]G1 | GPB6 | 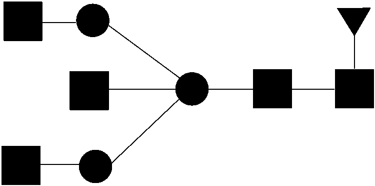 |
| | | | 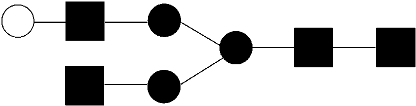 |
| 7 | A2[3]G1 F(6)A2B | GPB7 | 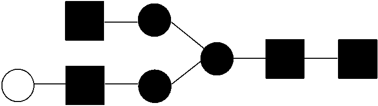 |
| | | | 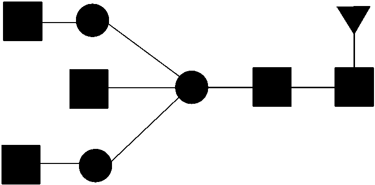 |
| 8 | A2BG1 F(6)A2[6]G1 | GPB8a | 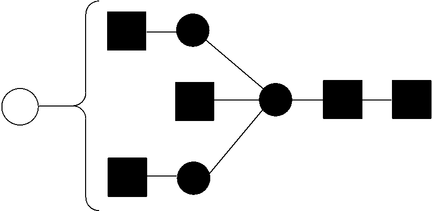 |
| | | | 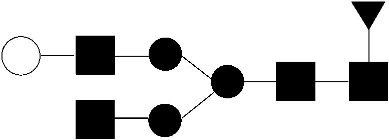 |
| 9 | F(6)A2[6]G1 | GPB8b | 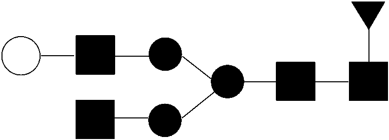 |
| 10 | F(6)A2[3]G1 | GPB9 | 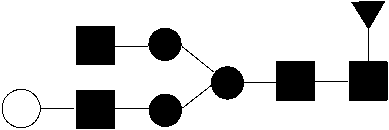 |
| 11 | F(6)A2[6]BG1 | GPB10 | 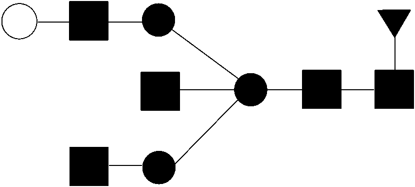 |
| 12 | F(6)A2[3]BG1 | GPB11 | 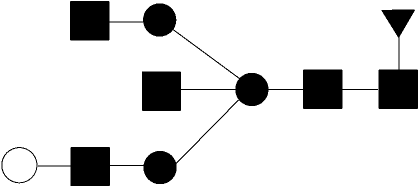 |
| 13 | A2G2 F(6)A2[3]BG1 | GPB12 | 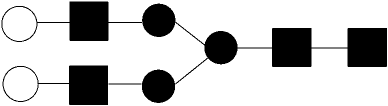 |
| | | | 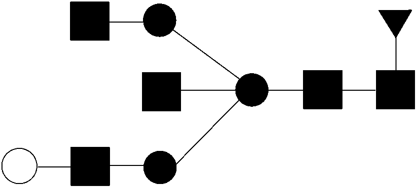 |
| 14 | A2BG2 F(6)A2G2 | GPB13 | 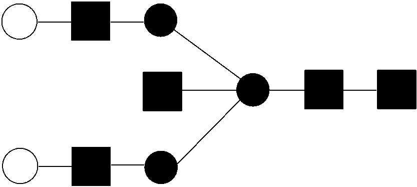 |
| | | | 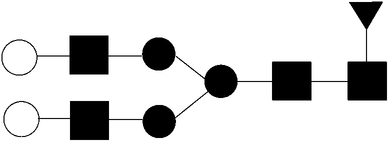 |
| 15 | F(6)A2G2 | GPB14 | 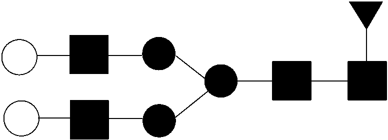 |
| 16 | F(6)A2BG2 F(6)A1G1S1 A2G1S1 F(6)A2G2 | GPB15 | 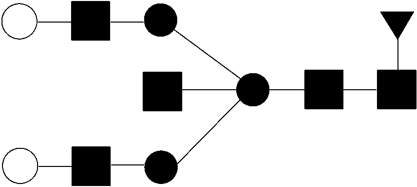 |
| | | | 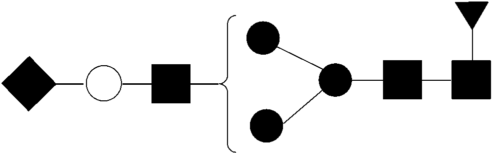 |
| | | | 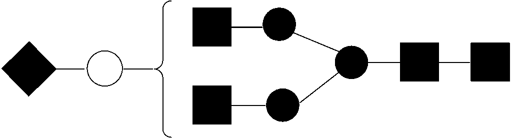 |
| | | | 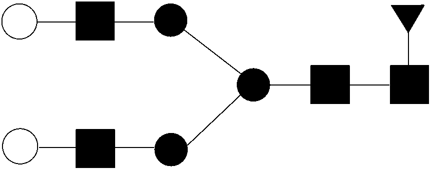 |
| 17 | F(6)A2[6]G1S1 M4A1G1S1 A2BG1S1 | GPB16a | 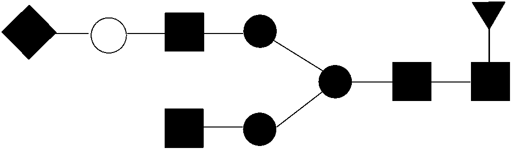 |
| | | | 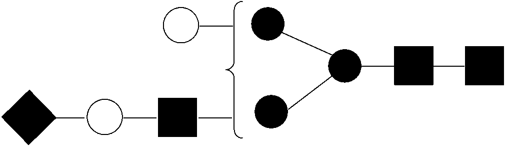 |
| | | | 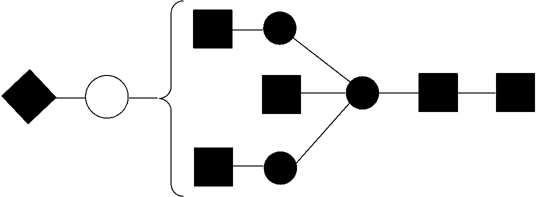 |
| 18 | F(6)A2[3]G1S1 F(6)A2[6]BG1S1 | GPB16b | 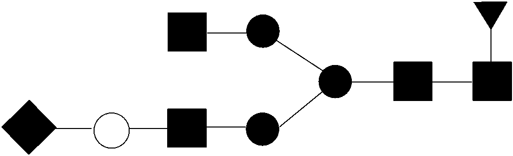 |
| | | | 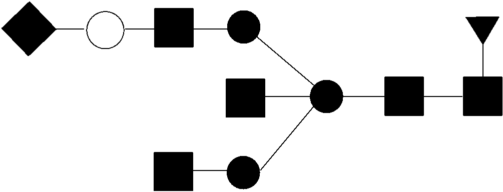 |
| 19 | A2G2S1 F(6)A2[3]BG1S1 | GPB17 | 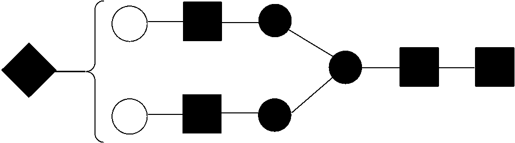 |
| | | | 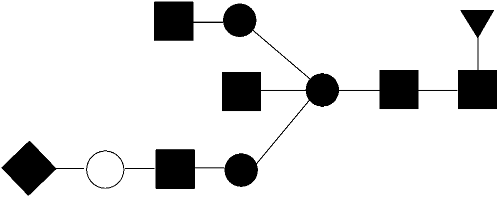 |
| 20 | A2BG2S2 F(6)A2G2S1 | GPB18a | 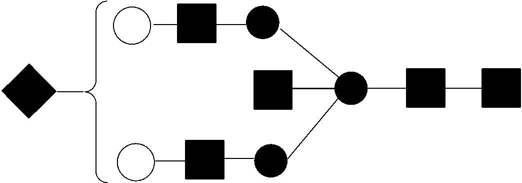 |
| | | | 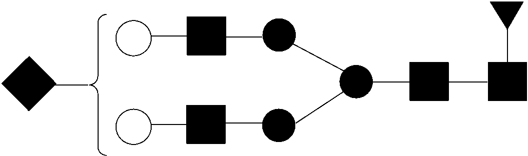 |
| 21 | F(6)A2G2S1 | GPB18b | 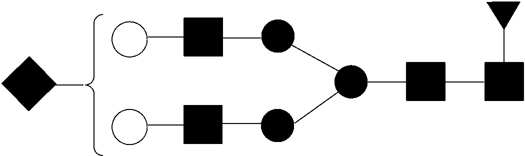 |
| 22 | F(6)A2BG2S1 | GPB19 | 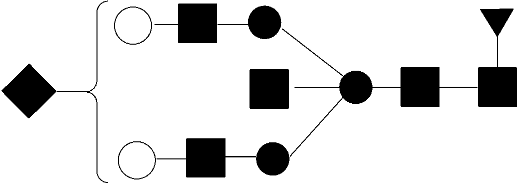 |
| 24 | A2G2S2 | GPB21 | 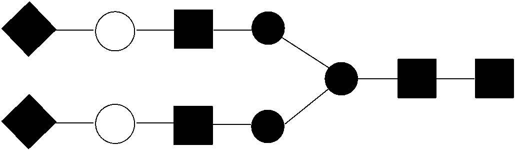 |
| 25 | A2BG2S2 | GPB22 | 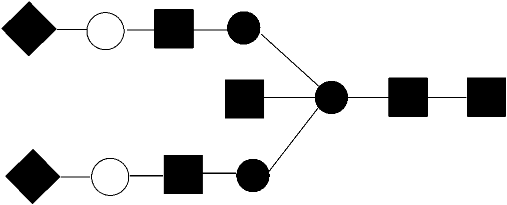 |
| 26 | F(6)A2G2S2 | GPB23 | 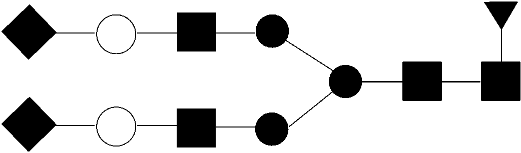 |
| 27 | F(6)A2BG2S2 | GPB24 | 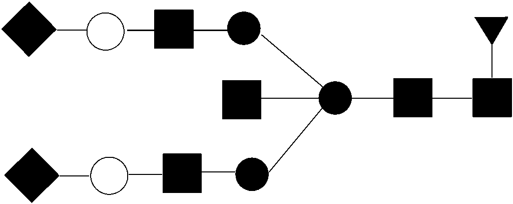 |

6. The process according to claim 1, wherein the set of glycans I, released from IgG, is further fluorescently derivatized in the step c) with alternative reagent 5-dioxopyrrolidine-1-yl-[2NV-(2-(N′,N′-diethylamino)ethyl)carbamoyl]-quinoline-6-yl-carbamate <RF>: 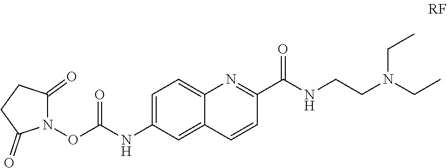 [SMILES: CCN(CC)CCNC(=O)c1ccc2cc(NC(=O)ON3C(=O)CCC3=O)ccc2n1]
and the resulting mixture is analyzed by ultra-performance liquid chromatography <UPLC>.

7. The process according to claim 5, wherein the logarithm of multiday average molar concentration of estradiol <E2>, is calculated from the following numerical model:
Log c<E2>=−15.529·GPB4−2.602·GPB8+5.589·GPB10+9.699·GPB12+53.911·GPB15+9.901·GPB16−1.990·GPB2·GPB10−0.065·GPB2·GPB12+3.601·GPB2·GPB15+0.007·GPB2·GPB16+0.465·(GPB4)^{2+2.889}·GPB4·GPB8+5.106·GPB4·GPB10−0.817·GPB4·GPB12−8.606·GPB4·GPB15+1.490·GPB4·GPB18+1.689·(GPB8)²−9.048·GPB8·GPB10−0.999·GPB8·GPB12−2.253·GPB8·GPB15+3.143·(GPB10)²+0.712·GPB10·GPB12−3.505·GPB10·GPB15−4.753·GPB10·GPB16+1.128·GPB10·GPB18−4.584·GPB12·GPB15+1.138·GPB12·GPB16−1.355·GPB12·GPB18−0.598·GPB12·GPB22−0.904·GPB12·GPB23−4.638·(GPB15)²+0.287·GPB15·GPB16−3.049·GPB15·GPB18+2.492·(GPB16)²−3.041·GPB16·GPB18
wherein factors GPB2, GPB4, GPB8, GPB10, GPB12, GPB15, GPB16, GPB18, GPB22 and GPB23 represent natural logarithms of corresponding values belonging to relative areas under the peaks of the respective glycans GPB2, GPB4, GPB8, GPB10, GPB12, GPB15, GPB16, GPB18, GPB22, GPB23 obtained from the chromatogram given by the selected quantitative analytical technique, and from which the multiday average molar concentration of estradiol c<E2> is calculated and expressed in picomoles per liter <pmol/L>.

8. The process according to claim 7, wherein multiday average molar concentration of estradiol c<E2> is interpreted as:
(a) c<E2> from 7 to 80, then the female subject has passed through the perimenopause phase and entered into menopause; or
(b) c<E2> from 80 to 800, then the female subject has not yet passed the perimenopause and thus has not entered menopause.

9. The process according to claim 1, wherein the selected model gives the numerical data indicative of the multiday average estradiol <E2> molar concentration in the blood of the examined female subject for a 3-month period.

10. The process according to claim 1, further comprising determination whether the examined female subject has passed through perimenopause and entered into menopause based on the multiday average estradiol <E2> molar concentration in the blood of the examined female subject.

11. The process according to claim 1, further comprising determination whether the examined female subject has entered into perimenopause based on the multiday average estradiol <E2> molar concentration in the blood of the examined female subject.
